(19) United States
(12) Patent Application Publication
(10) Pub. No.: US 2023/0008221 A1
(43) Pub. Date: Jan. 12, 2023

(54) PROCESS FOR PREPARING A MOLDING COMPRISING A ZEOLITE CATALYST AND METHOD FOR CONVERTING OXYGENATES TO OLEFINS USING THE CATALYTIC MOLDING

(71) Applicant: BASF SE, Ludwigshafen am Rhein (DE)

(72) Inventors: Andrei-Nicolae PARVULESCU, Ludwigshafen am Rhein (DE); Trees Maria DE BAERDEMAEKER, Ludwigshafen am Rhein (DE); Hamideh AHI, Ludwigshafen am Rhein (DE); Nakul THAKAR, Heidelberg (DE); Stefan KOTREL, Ludwigshafen am Rhein (DE); Emma Sian THOMPSON, Ludwigshafen am Rhein (DE); Matthias KELLERMEIER, Ludwigshafen am Rhein (DE); Ulrich MUELLER, Neustadt (DE)

(21) Appl. No.: 17/783,004

(22) PCT Filed: Dec. 10, 2020
(86) PCT No.: PCT/EP2020/085441

§ 371 Date: Jun. 7, 2022

(30) Foreign Application Priority Data
Dec. 11, 2019 (EP) 19215236.1

(51) Int. Cl. B01J 29/40; B01J 21/04; B01J 37/00; C07C 1/22

(52) U.S. Cl. CPC B01J 29/40; B01J 21/04; B01J 37/0018; C07C 1/22; C07C 2529/40; C07C 2521/04; C07C 2521/10

(57) ABSTRACT

The present invention relates to a process for preparing a molding comprising a zeolitic material and one or more oxidic binders, the process particularly comprising preparing a mixture of a zeolitic material, such as Mg-ZSM-5, a source of an oxidic binder, and a first plasticizer; admixing an acid to said mixture; and shaping of the mixture, to obtain a precursor of a molding; wherein in said mixture a specific weight ratio of the source of the oxidic binder to the sum of the zeolitic material and the source of the oxidic binder is applied. Further, the present invention relates to a molding obtainable or obtained by the inventive process, and to a molding itself displaying in particular a comparatively improved crush strength. Yet further, the present invention relates to a method for the conversion of oxygenates to olefins and to a use of the inventive molding.

### TECHNICAL FIELD

The present invention relates to a specific process for preparing a molding comprising use of a plasticizer, the molding prepared according to said process and its use. The molding exhibiting particular physical and chemical properties, while comprising a comparatively low amount of binder.

### INTRODUCTION

In the field of olefins syntheses, in particular the conversion of methanol to propylene plays an important role with increasing interest due to increasing availability of C1 starting materials. It is known that the synthesis in particular of short-chain olefins requires highly specific catalysts for converting the respective starting materials.

A particular challenge involved in such processes not only relies in the optimal choice of reaction parameters but, more importantly, in the use of particular catalysts allowing for the highly efficient and selective conversion for example to a desired olefinic fraction. As mentioned above, processes in which methanol is employed as the starting material, are of particular importance, wherein their catalytic conversion usually leads to a mixture of hydrocarbons and derivatives thereof, in particular olefins, paraffins, and aromatics.

Thus, the particular challenge in such catalytic conversions resides in the optimization and the fine tuning of the catalysts employed as well as the process architecture and parameters. Processes which have been developed in the past decades towards the conversion of oxygenates to olefins and in particular of methanol to olefins which have gained increasing importance in view of dwindling oil reserves are accordingly designated as methanol-to-olefin-processes (MTO-processes for methanol to olefins). Optimization of such processes is presently also of interest for reducing carbon dioxide emissions. Among the catalytic materials which have been found for use in such conversions, zeolitic materials have proven of high efficiency, wherein in particular zeolitic materials are employed.

WO 2012/085154 A1 relates to a process for preparing unsaturated carbon hydrates in the presence of a catalyst comprising a titanium-silicon-aluminum-phosphate (also designated as TAPSO therein) or a titanium-aluminum-phosphate. It is disclosed therein that the used titanium-silicon-aluminum-phosphate preferably has a CHA framework structure type.

US 2014/0058180 A1 relates to a process for producing a phosphorus-containing catalyst, wherein the catalyst comprises a zeolite, preferably having a TON, MTT, MFI, MEL, MTW, or EUO framework structure type. It is disclosed therein that the process comprises treating the calcined zeolite with an aqueous solution or water, whereby the aqueous solution can be selected from the group consisting of water, aqueous ammonium chloride, dilute hydrochloric acid, dilute acetic acid and dilute nitric acid. In particular, US 2014/058180 A1 discloses in Example 2 a method for preparing a molding comprising a zeolite and a binder wherein the weight ratio of the binder to the sum of the binder and the zeolite is about 0.176.

U.S. Pat. No. 10,112,188 B2 and US 2014/0058181 A1 relate to a process for preparing a phosphorus containing zeolite type catalyst based on crystalline aluminosilicates, the catalyst of said process and the use of said catalysts for the conversion of methanol to olefins. The process comprises mixing an aluminum oxide and an acid to a zeolite powder of pentasil, wherein the acid can be sulfuric acid, nitric acid, acetic acid, formic acid, oxalic acid or citric acid.

U.S. Pat. No. 10,005,073 B2 also relates to the production of a phosphorous containing zeolite preferably having an MFI or MEL framework structure type.

U.S. Pat. No. 9,511,361 B2 relates to a catalyst containing a pentasil-type alumosilicate and a binder, wherein the catalyst is in the form of spheres having a specific average diameter and a specific BET surface area. The catalyst may be used for the conversion of methanol to olefins. Further, it is disclosed that the prepared catalyst may comprise 10 to 40 weight-% of binder, relative the total weight of aluminosilicate and binder.

US 2017/0121259 A1 relates to a process for producing a catalyst containing copper, zinc and aluminum, in particular for producing catalyst moldings having increased mechanical strength, in particular a lateral compressive strength.

WO 2018/109083 A1 relates to a tableted catalyst for methanol synthesis having increased mechanical stability. The catalyst comprises a metal-containing mixture, containing copper, zinc, and aluminum, with calcium aluminate as a binder material.

CN 100503041 C relates to a catalyst for dimethyl ether synthesis and preparation method thereof, wherein the catalyst comprises a hydrophobic zeolite having a proton, a cation selected from alkali metals, alkaline earth metals and ammonium, and an inorganic binder selected from alumina, silica, and silica-alumina. Preparation of said catalysts would include providing a paste prepared from a mixture of an acid and a binder, mixing said paste with a zeolite and extruding of the resulting mixture.

CN 104511298 B relates to a catalyst system for the conversion of methanol to propylene, wherein the catalyst is characterized in that it contains, based on the total weight of the catalyst system, a) 30-85 weight-% of a modified zeolite molecular sieve having an SAR of 100-3000, b) 0.001-5 weight-% of improver, c) 0.1-20 weight-% of a co-catalyst component, d) 10-50 weight-% of a hydrophobic silicon powder, and e) 3-55 weight-% of binder.

Despite the considerable efforts related by the prior art relative to the synthesis of novel catalytic materials on the one hand by using new and improved synthetic procedures, and their various applications such as in particular in the field of catalysis on the other hand, there remains an ongoing need to provide new catalytic materials, in particular specific moldings, displaying yet further improved properties in particular with respect to their mechanical stability to achieve an improved longevity.

### DETAILED DESCRIPTION

Thus, there remains still the need for providing a preparation process of a molding showing improved mechanical properties while keeping excellent catalytic activity in the conversion of oxygenates to olefins, in particular for the conversion of methanol to propylene. It is of particular interest to provide such a molding showing an improved mechanical stability, in particular an improved crush strength, while keeping an excellent catalytic activity.

Accordingly, it was an object of the present invention to provide a novel molding suitable for the conversion of oxygenates to olefins, in particular for the selective conversion of methanol to propylene, for example under fixed application, having comparatively improved physical properties, in particular an improved mechanical strength. Similarly, a molding can be prepared according to the novel process having an improved tortuosity with respect to water, and an improved diffusion coefficient. Thus, it was a further object of the present invention to provide a process for preparing such a molding, in particular a process having comparatively reduced carbon dioxide emissions.

Surprisingly, it has presently been found that a novel process for preparing a molding can be provided resulting in a molding having improved mechanical properties. Furthermore, it has surprisingly been found that a molding can be prepared according to the present invention displaying improved physical and chemical properties, in particular an improved mechanical strength and also an improved tortuosity with respect to water, and an improved diffusion coefficient. The molding of the present were also shown to achieve an excellent catalytic activity in the conversion of methanol to olefins.

Therefore, the present invention relates to a process for preparing a molding comprising a zeolitic material and one or more oxidic binders, wherein the zeolitic material comprises YO₂ and optionally X₂O₃ in its framework structure,

wherein Y is a tetravalent element and X is a trivalent element, the process comprising
(i) preparing a mixture comprising a zeolitic material, a source of an oxidic binder, a first plasticizer, and an acid;
(ii) preferably admixing water to the mixture obtained in (i);
(iii) preferably admixing a second plasticizer to the mixture obtained in (i) or (ii);
(iv) preferably admixing water to the mixture obtained in (iii);
(v) shaping of the mixture obtained in (i), (ii), (iii) or (iv), to obtain a precursor of a molding;
wherein in the mixture obtained in (i) the weight ratio of the source of the oxidic binder, calculated as the oxide, to the sum of the zeolitic material and the source of the oxidic binder, calculated as the oxide, is in the range of from 0.05:1 to 0.15:1.

According to the present invention, a molding is to be understood as a three-dimensional entity obtained from a shaping process; accordingly, the term “molding” is used synonymously with the term “shaped body”.

Typically, the zeolitic material comprised in the molding of the present invention is in the form of a powder which, as to its particle size distribution, can be prepared, for example, by a specific synthesis process leading to the desired particle size distribution, or by milling a given zeolitic material, or by spray-drying a suspension comprising a zeolitic material, or by spray-granulation of a suspension comprising a zeolitic material, or by flash drying a suspension comprising a zeolitic material or by microwave drying a suspension comprising a zeolitic material.

Preferably, (i) of the process comprises according to a first alternative

(i.1.a) providing a mixture comprising a zeolitic material, a source of an oxidic binder, and a first plasticizer;
(i.1.b) admixing an acid to the mixture obtained in (i.1.a).

Preferably, (i) of the process comprises according to a second alternative

(i.2.a) providing a zeolitic material;
(i.2.b) providing a mixture comprising a source of an oxidic binder, an acid, and optionally water;
(i.2.c) mixing the mixture obtained in (i.2.b) with the zeolitic material provided in (i.2.a);
(i.2.d) admixing a first plasticizer to the mixture obtained in (i.2.c).

In the case where the process comprises (i.2.c), mixing according to (i.2.c) may be carried out by admixing the mixture provided in (i.2.b) to the zeolitic material provided in (i.2.a) or admixing the zeolitic material provided in (i.2.a) to the mixture provided in (i.2.b).

In the case where the process comprises (i.2.a), (i.2.b), (i.2.c), and (i.2.d) according to the second alternative, it is preferred that water is comprised in the mixture obtained in (i.2.b), wherein the mixture obtained in (i.2.b) more preferably exhibits a weight ratio of water to the source of the oxidic binder, calculated as source of the oxidic binder as such, in the range of from 1:1 to 10:1, more preferably in the range of from 4.0:1 to 5.0:1, more preferably in the range of from 4.50:1 to 4.70:1

More preferably, the present invention relates to a process for preparing a molding comprising a zeolitic material and one or more oxidic binders, wherein the zeolitic material comprises YO₂ and optionally X₂O₃ in its framework structure,

wherein Y is a tetravalent element and X is a trivalent element, the process comprising
(i′) preparing a mixture of a zeolitic material, a source of an oxidic binder, and a first plasticizer;
(ii′) admixing an acid to the mixture obtained from (i′);
(iii′) preferably admixing water to the mixture obtained from (ii′);
(iv′) preferably admixing a second plasticizer to the mixture obtained from (ii′) or (iii′);
(v′) preferably admixing water to the mixture obtained from (iv′);
(vi′) shaping of the mixture obtained from (ii′), (iii′), (iv′) or (v′), to obtain a precursor of a molding;
wherein in the mixture prepared in (i′) the weight ratio of the source of the oxidic binder, calculated as the oxide, to the sum of the zeolitic material and the source of the oxidic binder, calculated as the oxide, is in the range of from 0.05:1 to 0.15:1.

It is preferred that the mixture prepared in (i) or (i′) is mixed in a kneader, Lodige mixer (German: Lödige Mischer) or mix-muller.

No particular restriction applies, with respect to the content of the first plasticizer in the mixture prepared in (i) or (i′). It is preferred that in the mixture prepared in (i) or (i′) the weight ratio of the first plasticizer to the sum of the zeolitic material and the source of the oxidic binder, calculated as source of the oxidic binder as such, is in the range of from 0.01:1 to 0.1:1, preferably in the range of from 0.02:1 to 0.08:1, more preferably in the range of from 0.03:1 to 0.07:1, more preferably in the range of from 0.04:1 to 0.06:1, more preferably in the range of from 0.045:1 to 0.055:1.

Generally, any suitable compound may be chosen as first plasticizer as long as it fulfills its function. It is preferred that the first plasticizer is an organic compound. It is particularly preferred that the first plasticizer is selected from the group consisting of organic polymers, carbohydrates, graphite, plant additives, and mixtures of two or more thereof, more preferably from the group consisting of polymeric vinyl compounds, polyalkylene oxides, polyacrylates, polymethacrylates, polyolefins, polyamides, polyesters, polystyrenes, polysaccharides, and mixtures of two or more thereof, wherein the first plasticizer is more preferably a polysaccharide.

In the case where the first plasticizer is a polysaccharide, it is preferred that the polysaccharide is selected from the group consisting of celluloses, cellulose derivatives, and starches, wherein the polysaccharide more preferably is one or more of methyl cellulose and carboxmethylcellulose.

Further in the case where the first plasticizer is a polysaccharide, it is preferred that the polysaccharide has a bulk density in the range of from 500 to 800 g/l, more preferably in the range of from 550 to 750 g/l, more preferably in the range of from 600 to 700 g/l, more preferably in the range of from 630 to 670 g/l.

Further in the case where the first plasticizer is a polysaccharide, it is preferred that the polysaccharide has a viscosity in the range of from 3000 to 4000 mPas, more preferably in the range of from 3400 to 3600 mPas, more preferably in the range of from 3450 to 3550 mPas.

No particular restriction applies with respect to the content of the source of the oxidic binder. It is preferred that the weight ratio of the source of the oxidic binder, calculated as the oxide, to the sum of the zeolitic material and the source of the oxidic binder, calculated as the oxide, in the mixture obtained in (i) or (i′), is in the range of from 0.06:1 to 0.14:1, more preferably in the range of from 0.07:1 to 0.13:1, more preferably in the range of from 0.08:1 to 0.12:1 more preferably in the range of from 0.09:1 to 0.11:1.

Generally, any suitable compound may be used as a source of the oxidic binder. It is preferred that the source of the oxidic binder is a source of one or more of silica, alumina, and silica-alumina, more preferably a source of alumina.

It is particularly preferred that the source of the oxidic binder comprises one or more of AlOOH (boehmite), Al₂O₃, Al(OH)₃, hydrotalcite, a silica sol, a colloidal silica, a wet process silica, and a dry process silica, preferably one or more of AlOOH (boehmite) and Al₂O₃, more preferably AlOOH (boehmite), wherein the source of the oxidic binder more preferably is AlOOH (boehmite).

As regards the alternative according to which the oxidic binder may be a silica, both colloidal silica and so-called “wet process” silica and so-called “dry process” silica can be used.

No particular restriction with respect to the tetravalent element Y of the zeolitic material, such that any tetravalent element of the periodic system of elements may be used for Y. It is preferred that Y is selected from the group consisting of Si, Sn, Ti, Zr, Ge, and a mixture of two or more thereof, more preferably from the group consisting of Si, Ti, and a mixture thereof, wherein more preferably Y is Si.

No particular restriction with respect to the trivalent element X of the zeolitic material, such that any trivalent element of the periodic system of elements may be used for X. It is preferred that X is selected from the group consisting of B, Al, Ga, In, and a mixture of two or more thereof, more preferably from the group consisting of B, Al and a mixture thereof, wherein more preferably X is Al.

No particular restriction applies with respect to the molar ratio of YO₂ to X₂O₃ of the zeolitic material. It is preferred that the zeolitic material has a molar ratio of YO₂ to X₂O₃ in the range of from 50 to 150, more preferably in the range of from 75 to 125, more preferably in the range of from 90 to 120, more preferably in the range of from 95 to 115. It is particularly preferred that the molar ratio of YO₂ to X₂O₃ of the zeolitic material is a molar silica to alumina ratio.

No restriction applies with respect to the framework structure type of the zeolitic material. It is preferred that the zeoltic material has a framework structure type selected from the group consisting of ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE, BSV, CAN, CAS, CDO, CFI, CGF, CGS, CHA, -CHI, -CLO, CON, CSV, CZP, DAC, DDR, DFO, DFT, DOH, DON, EAB, EDI, EEI, EMT, EON, EPI, ERI, ESV, ETR, EUO, *-EWT, EZT, FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH, *-ITN, ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MFI, MFS, MON, MOR, MOZ, *MRE, MSE, MSO, MTF, MTN, MTT, MTW, MVY, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF, *-SSO, SSY, STF, STI, *STO, STT, STW, -SVR, SW, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, and mixed types of two or more thereof, more preferably from the group consisting of MFI, MEL, ITH, IWR, CON, and mixed types of two or more thereof, more preferably selected from the group consisting of MFI, ITH, IWR, CON, and mixed types of two or more thereof. It is particularly preferred that the zeolitic material has the MFI framework structure type.

In the case where the zeolitic material has the MFI framework structure type, it is preferred that the zeolitic material is selected from the group consisting of Silicalite, ZSM-5, [Fe—Si—O]-MFI, [Ga—Si—O]-MFI, [As—Si—O]-MFI, AMS-1B, AZ-1, Bor-C, Encilite, Boralite C, FZ-1, LZ-105, Mutinaite, NU-4, NU-5, TS-1, TSZ, TSZ-III, TZ-01, USC-4, USI-108, ZBH, ZKQ-1B, ZMQ-TB, MnS-1, and FeS-1, including mixtures of two or more thereof, more preferably from the group consisting of Silicalite, ZSM-5, AMS-1B, AZ-1, Encilite, FZ-1, LZ-105, Mutinaite, NU-4, NU-5, TS-1, TSZ, TSZ-III, TZ-01, USC-4, USI-108, ZBH, ZKQ-1B, and ZMQ-TB, including mixtures of two or more thereof, wherein more preferably the zeolitic material having an MFI-type framework structure comprises Silicalite and/or ZSM-5, preferably ZSM-5, wherein more preferably the zeolitic material having an MFI-type framework structure is zeolite Silicalite and/or ZSM-5, preferably ZSM-5.

It is preferred that the zeolitic material comprises one or more alkaline earth metals M, wherein the one or more alkaline earth metals M preferably are selected from the group consisting of Be, Mg, Ca, Sr, Ba, and a mixture of two or more thereof, preferably from the group consisting of Mg, Ca, and a mixture thereof, wherein more preferably the alkaline earth metal M comprises, more preferably is, Mg.

In the case where the zeolitic material comprises one or more alkaline earth metals M, it is preferred that the zeolitic material comprises the alkaline earth metal M in an amount in the range of from 0.5 to 4.0 weight-%, calculated as the element, based on the weight of the molding, more preferably in the range of from 1.0 to 3.0 weight-%, more preferably in the range of from 1.5 to 2.7 weight-%, more preferably in the range of from 1.7 to 2.5 weight-%.

Further in the case where the zeolitic material comprises one or more alkaline earth metals M, it is preferred that the zeolitic material comprises the alkaline earth metal M in an amount in the range of from 0.5 to 4.0 weight-%, calculated as the element, based on the weight of the zeolitic material, more preferably in the range of from 1.8 to 2.6 weight-%, more preferably in the range of from 2.0 to 2.4 weight-%, more preferably in the range of from 2.1 to 2.3 weight-%.

It is preferred that the zeolitic material is impregnated with the one or more alkaline earth metals M.

No particular restriction applies with respect to the method according to which the zeolitic material is impregnated with the one or more alkaline earth metals M. In the case where the zeolitic material is impregnated with the one or more alkaline earth metals M, it is preferred that the zeolitic material is impregnated with the one or more alkaline earth metals M by spray-impregnation, adhesion impregnation, incipient impregnation, or wet impregnation adhesion technique.

No particular restriction applies with respect to the weight ratio of the source of the oxidic binder to the zeolitic material in the mixture prepared in (i) or (i′). It is preferred that in the mixture prepared in (i) or (i′) the weight ratio of the source of the oxidic binder, calculated as source of the oxidic binder as such, to the zeolitic material is in the range of from 0.05:1 to 0.25:1, more preferably in the range of from 0.07:1 to 0.22:1, more preferably in the range of from 0.10:1 to 0.19:1, more preferably in the range of from 0.11:1 to 0.18:1, more preferably in the range of from 0.12:1 to 0.17:1, more preferably in the range of from 0.13:1 to 0.16:1, more preferably in the range of from 0.14:1 to 0.15:1.

No particular restriction applies with respect to the chemical or physical nature of the acid admixed in (ii′) or comprised in the mixture prepared in (i). It is preferred that the acid is one or more of an inorganic acid and an organic acid. In the case where the acid comprises an organic acid, it is preferred that the organic acid is one or more of formic acid, acetic acid, propionic acid, oxalic acid, and tartaric acid, more preferably formic acid. In the case where the acid comprises an inorganic acid, it is preferred that the inorganic acid is one or more of hydrochloric acid, nitric acid, and phosphoric acid, more preferably nitric acid. It is particularly preferred that the acid comprises, preferably is, one or more of formic acid and nitric acid.

According to a first alternative, it is preferred that the acid is admixed in (i.1.b) or provided for providing the mixture in (i.2.b) as an aqueous solution, more preferably as an aqueous solution comprising an amount of the acid in the range of from 5 to 50 weight-%, more preferably in the range of from 10 to 40 weight-%, more preferably in the range of from 12.5 to 37.5 weight-%, more preferably in the range of from 15 to 35 weight-%, more preferably in the range of from 17.5 to 32.5 weight-%, more preferably in the range of from 20 to 30 weight-%, more preferably in the range o from 22.5 to 27.5 weight-%, more preferable in the range of from 24 to 26 weight-%, based on the totalweight of the aqueous solution, wherein the acid is preferably formic acid.

According to a second alternative, it is preferred that the acid is admixed in (i.1.b) or provided for providing the mixture in (i.2.b) as an aqueous solution, more preferably as an aqueous solution comprising an amount of the acid in the range of from 1 to 20 weight-%, more preferably in the range of from 3 to 15 weight-%, more preferably in the range of from 5 to 13 weight-%, more preferably in the range of from 6 to 12 weight-%, more preferably in the range of from 7 to 11 weight-%, more preferable in the range of from 8 to 10 weight-%, based on the total weight of the aqueous solution, wherein the acid is preferably nitric acid.

In the case where the first or the second alternative apply with respect to admixing the acid in (i.1.b) or providing the acid for providing the mixture in (i.2.b), it is preferred that the weight ratio of the acid admixed in (i.1.b) or provided for providing the mixture in (i.2.b) to the sum of the zeolitic material and the source of the oxidic binder of the mixture prepared in (i) or (i′) is in the range of from 0.05:1 to 0.15:1, more preferably in the range of from 0.06:1 to 0.14:1, preferably in the range of from 0.07:1 to 0.13:1, more preferably in the range of from 0.08:1 to 0.12:1 more preferably in the range of from 0.09:1 to 0.11:1.

According to a third alternative, it is preferred that the acid is admixed in (i.1.b) or provided for providing the mixture in (i.2.b) as an aqueous solution, preferably as an aqueous solution comprising an amount of the acid in the range of from 50 to 80 weight-%, more preferably in the range of from 55 to 75 weight-%, more preferably in the range o from 60 to 70 weight-%, more preferable in the range of from 63 to 67 weight-%, based on the total weight of the aqueous solution, wherein the acid is preferably nitric acid.

In the case where the third alternative apply, it is preferred that the weight ratio of the acid admixed in (i.1.b) or provided for providing the mixture in (i.2.b) to the sum of the zeolitic material and the source of the oxidic binder of the mixture prepared in (i) or (i′) is in the range of from 0.005:1 to 0.05:1, more preferably in the range of from 0.010:1 to 0.030:1 more preferably in the range of from 0.015:1 to 0.025:1.

It is preferred that in (ii) or (iii′) water is admixed to the mixture obtained from, preferably in, (i) or (ii′), preferably de-ionized water.

In the case where in (ii) or (iii′) water is admixed to the mixture obtained from, preferably in, (i) or (ii′), it is preferred that in the mixture in (ii) or (iii′) the weight ratio of water to the sum of the source of the oxidic binder and the zeolitic material is in the range of from 0.1:1 to 1.5:1, more preferably in the range of from 0.2:1 to 0.8:1, more preferably in the range of from 0.3:1 to 0.7:1, more preferably in the range of from 0.3:1 to 0.6:1, more preferably in the range of from 0.4:1 to 0.5:1 more preferably in the range of from 0.45:1 to 0.46:1.

It is preferred that in (iii) or (iv′) a second plasticizer is admixed to the mixture obtained from, preferably in, (i), (ii), (ii′) or (iii′), wherein the second plasticizer preferably is different to the first plasticizer.

In the case where a second plasticizer is admixed to the mixture obtained from, preferably in, (i), (ii), (ii′) or (iii′), it is preferred that in the mixture in (iii) or (iv′) the weight ratio of the second plasticizer to the sum of the source of the oxidic binder and the zeolitic material is in the range of from 0.001:1 to 0.030:1, more preferably in the range of from 0.005:1 to 0.015:1, more preferably in the range of from 0.007:1 to 0.013:1, more preferably in the range of from 0.008:1 to 0.012:1 more preferably in the range of from 0.009:1 to 0.011:1.

No particular restriction applies with respect to the chemical or physical nature of the second plasticizer. In the case where a second plasticizer is admixed to the mixture obtained from, preferably in, (i), (ii), (ii′) or (iii′), it is preferred that the second plasticizer is an organic compound. It is particularly preferred that the second plasticizer is selected from the group consisting of organic polymers, carbohydrates, graphite, plant additives, and mixtures of two or more thereof, more preferably from the group consisting of polymeric vinyl compounds, polyalkylene oxides, polyacrylates, polymethacrylates, polyolefins, polyamides, polyesters, polystyrenes, polysaccharides, and mixtures of two or more thereof. It is particularly preferred that the second plasticizer is a polyethylene oxide or a polysaccharide.

In the case where the second plasticizer is a polysaccharide, it is preferred that the polysaccharide is selected from the group consisting of celluloses, cellulose derivatives, and starches, wherein the polysaccharide is more preferably one or more of methyl cellulose and carboxmethylcellulose.

It is preferred that in (iv) or (v′) water is admixed to the mixture obtained from, preferably in, (i), (ii), (iii), (ii′), (iii′), or (iv′), preferably de-ionized water.

In the case where in (iv) or (v′) water is admixed to the mixture obtained from, preferably in, (i), (ii), (iii), (ii′), (iii′), or (iv′), it is preferred that in the mixture in (iv) or (v′) the weight ratio of water to the sum of the source of the oxidic binder and the zeolitic material is in the range of from 0.1:1 to 1:1, more preferably in the range of from 0.30:1 to 0.90:1, more preferably in the range of from 0.50:1 to 0.7:1, more preferably in the range of from 0.55:1 to 0.65:1 more preferably in the range of from 0.60:1 to 0.61:1.

It is preferred that in (v) or (vi′), the mixture is shaped to a strand, more preferably having a hexagonal, rectangular, quadratic, triangular, oval, or circular cross-section, more preferably having a circular cross-section.

In the case where the strand has a circular cross-section, it is preferred that the strand having a circular cross-section has a diameter in the range of from 0.5 to 7 mm, more preferably in the range of from 1.5 to 3.5 mm, more preferably in the range of from 2.1 to 2.9 mm, more preferably in the range of from 2.3 to 2.7 mm, more preferably in the range of from 2.4 to 2.6 mm.

It is preferred that in (v) or (vi′), shaping comprises extruding the mixture.

Suitable extrusion apparatuses are described, for example, in “Ullmann's Enzyklopädie der Technischen Chemie”, 4th edition, vol. 2, page 295 et seq., 1972. In addition to the use of an extruder, an extrusion press can also be used for the preparation of the moldings. If necessary, the extruder can be suitably cooled during the extrusion process. The strands leaving the extruder via the extruder die head can be mechanically cut, for example by a suitable wire or via a discontinuous gas stream.

It is preferred that shaping according to (v) or (vi′) further comprises drying the precursor of the molding in a gas atmosphere.

It is preferred that drying is carried out at a temperature of the gas atmosphere in the range of from 80 to 160° C., more preferably in the range of from 100 to 140° C., more preferably in the range of from 110 to 130° C. It is preferred that the gas atmosphere comprises nitrogen, oxygen, or a mixture thereof, wherein the gas atmosphere is more preferably oxygen, air, or lean air.

It is preferred that shaping according to (v) or (vi′) further comprises calcining the, preferably dried, precursor of the molding in a gas atmosphere. It is preferred that calcining is carried out at a temperature of the gas atmosphere in the range of from 500 to 650° C., more preferably in the range of from 530 to 570° C., more preferably in the range of from 540 to 560° C. It is preferred that the gas atmosphere comprises nitrogen, oxygen, or a mixture thereof, wherein the gas atmosphere is more preferably oxygen, air, or lean air.

Further, the present invention relates to a molding obtainable or obtained by the process of any one of the embodiments disclosed herein.

Further, the present invention relates to a molding, preferably prepared according to the process of any one of the embodiments disclosed herein, comprising one or more oxidic binders and a zeolitic material wherein the zeolitic material comprises YO₂ and optionally X₂O₃ in its framework structure,

wherein Y is a tetravalent element and X is a trivalent element,
wherein the molding comprises the one or more oxidic binders, calculated as the oxide, in an amount in the range of from 5 to 15 weight-%, and wherein the molding exhibits a crush strength of equal or greater than 9 N. It is preferred that the crush strength is determined according to Reference Example 5.

It is preferred that the molding exhibits a crush strength of equal or greater than 10 N, more preferably of equal or greater than 15 N, more preferably equal or greater than 18 N, more preferably equal or greater than 19 N, more preferably equal or greater than 20 N. It is preferred that the crush strength is determined according to Reference Example 5. In particular, it is preferred that the molding exhibits a crush strength in the range of from 15 to 50 N, more preferably in the range of from 17 to 30 N.

It is preferred that the molding exhibits a diffusion coefficient in the range of from 0.40 to 1.30×10⁻⁹ m²/s, more preferably in the range of from 0.60 to 1.10×10⁻⁹ m²/s, more preferably in the range of from 0.72 to 0.98×10⁻⁹ m²/s. It is preferred that the diffusion coefficient is determined according to Reference Example 4.

It is preferred that the molding exhibits a tortuosity parameter relative to water in the range of from 1.00 to 3.75, more preferably in the range of from 1.2 to 3.0, more preferably in the range of from 1.4 to 2.8. It is preferred that the tortuosity parameter relative to water is determined as described in Reference Example 2.

No particular restriction applies to the chemical or physical nature of the one or more oxidic binders comprised in the molding. It is preferred that the one or more oxidic binders are selected from the group consisting of silica, alumina, silica-alumina, and a mixture of two or more thereof, wherein the one or more oxidic binders preferably are alumina.

It is preferred that the molding comprises the one or more oxidic binders, calculated as the oxide, in an amount in the range of from 6 to 14 weight-%, more preferably in the range of from 7 to 13 weight-%, more preferably in the range of from 8 to 12 weight-%, more preferably in the range of from 9 to 11 weight-%.

No particular restriction with respect to the tetravalent element Y of the zeolitic material comprised in the molding, such that any tetravalent element of the periodic system of elements may be used for Y. It is preferred that Y is selected from the group consisting of Si, Sn, Ti, Zr, Ge, and a mixture of two or more thereof, more preferably from the group consisting of Si, Ti, and a mixture thereof, wherein more preferably Y is Si.

No particular restriction with respect to the trivalent element X of the zeolitic material comprised in the molding, such that any trivalent element of the periodic system of elements may be used for X. It is preferred that X is selected from the group consisting of B, Al, Ga, In, and a mixture of two or more thereof, more preferably from the group consisting of B, Al and a mixture thereof, wherein more preferably X is Al.

No restriction applies with respect to the framework structure type of the zeolitic material comprised in the molding. It is preferred that the zeolitic material comprised in the molding has a framework structure type selected from the group consisting of ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE, BSV, CAN, CAS, CDO, CFI, CGF, CGS, CHA, -CHI, -CLO, CON, CSV, CZP, DAC, DDR, DFO, DFT, DOH, DON, EAB, EDI, EEI, EMT, EON, EPI, ERI, ESV, ETR, EUO, *-EWT, EZT, FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH, *-ITN, ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MFI, MFS, MON, MOR, MOZ, *MRE, MSE, MSO, MTF, MTN, MTT, MTW, MVY, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF,*-SSO, SSY, STF, STI,*STO, STT, STW, -SVR, SW, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, and mixed types of two or more thereof, more preferably from the group consisting of MFI, MEL, ITH, IWR, CON, and mixed types of two or more thereof, more preferably selected from the group consisting of MFI, ITH, IWR, CON, and mixed types of two or more thereof. It is particularly preferred that the zeolitic material has the MFI framework structure type.

It is preferred that the zeolitic material comprised in the molding comprises X₂O₃. In the case where the zeolitic material comprised in the molding comprises X₂O₃, it is preferred that the zeolitic material has a molar ratio of YO₂ to X₂O₃ in the range of from 50 to 150, more preferably in the range of from 75 to 125, more preferably in the range of from 90 to 120, more preferably in the range of from 95 to 115.

In the case where the zeolitic material comprised in the molding has the framework structure type MFI, it is preferred that the zeolitic material having the MFI framework structure type comprises one or more of ZSM-5, ZBM-10, [As—Si—O]-MFI, [Fe—Si—O]-MFI, [Ga—Si—O]-MFI, AMS-1B, AZ-1, boron-C, boralite C, encilite, FZ-1, LZ-105, monoclinic H-ZSM-5, mutinaite, NU-4, NU-5, silicalite, TS-1, TSZ, TSZ-III, TZ-01, USC-4, USI-108, ZBH, ZKQ-1B, and ZMQ-TB, more preferably one or more of ZSM-5, and ZBM-10, more preferably ZSM-5.

It is preferred that from 99 to 100 weight-%, more preferably from 99.5 to 100 weight-%, more preferably from 99.9 to 100 weight-%, of the zeolitic material comprised in the molding consist of Y, optionally X, O, and H.

It is preferred that the zeolitic material comprised in the molding comprises one or more alkaline earth metals M.

In the case where the zeolitic material comprised in the molding comprises one or more alkaline earth metals M, it is preferred that the one or more alkaline earth metals M are selected from the group consisting of Be, Mg, Ca, Sr, Ba, and a mixture of two or more thereof, more preferably from the group consisting of Mg, Ca, and a mixture thereof, wherein more preferably the one or more alkaline earth metals M comprise, more preferably consist of, Mg.

Further in the case where the zeolitic material comprised in the molding comprises one or more alkaline earth metals M, it is preferred that the zeolitic material comprises the one or more alkaline earth metals M in an amount in the range of from 0.1 to 5 weight-%, calculated as element, based on the weight of the molding, more preferably in the range of from 1.5 to 2.5 weight-%, more preferably in the range of from 1.7 to 2.3 weight-%.

Further in the case where the zeolitic material comprised in the molding comprises one or more alkaline earth metals M, it is preferred that the zeolitic material comprises the one or more alkaline earth metals M in an amount in the range of from 0.5 to 4.0 weight-%, calculated as the element, based on the weight of the zeolitic material, more preferably in the range of from 1.0 to 3.0 weight-%, more preferably in the range of from 1.5 to 2.7 weight-%, more preferably in the range of from 1.7 to 2.5 weight-%, more preferably in the range of from 1.7 to 2.3 weight-%.

Further in the case where the zeolitic material comprised in the molding comprises one or more alkaline earth metals M, it is preferred that the zeolitic material is impregnated, preferably spray-impregnated, with the one or more alkaline earth metals M.

Further in the case where the zeolitic material comprised in the molding comprises one or more alkaline earth metals M, it is preferred that from 99 to 100 weight-%, more preferably from 99.5 to 100 weight-%, more preferably from 99.9 to 100 weight-%, of the zeolitic material consist of Y, optionally X, O, H, and the one or more alkaline earth metals M.

It is preferred that the molding comprises less than 1 weight-% of sodium, more preferably less than 0.1 weight-% of sodium, more preferably less than 0.01 weight-% of sodium.

It is preferred that the molding has a BET specific surface area in the range of from 300 to 400 m²/g, more preferably in the range of from 325 to 375 m²/g, more preferably in the range of from 350 to 360 m²/g. It is preferred that the BET specific surface area is determined as described in Reference Example 1.

It is preferred that the molding has a total pore volume in the range of from 0.2 to 0.75 g/ml, more preferably in the range of from 0.45 to 0.53 g/ml, more preferably in the range of from 0.47 to 0.51 g/ml, more preferably in the range of from 0.48 to 0.50 g/ml. It is preferred that the total pore volume is determined according to Reference Example 3.

It is preferred that the molding exhibits an acid site density in the range of from 0.20 to 0.75 mmol/g, more preferably in the range of from 0.25 to 0.65 mmol/g, more preferably in the range of from 0.44 to 0.52 mmol/g, more preferably in the range of from 0.46 to 0.50 mmol/g, more preferably in the range of from 0.47 to 0.49 mmol/g, at a temperature below 250° C. It is preferred that the acid site density is determined according to Reference Example 6.

It is preferred that the molding exhibits an acid site density of equal or smaller than 0.5 mmol/g, more preferably of equal or smaller than 0.30 mmol/g, more preferably of equal or smaller than 0.25 mmol/g, more preferably of equal or smaller than 0.1 mmol/g, more preferably of equal or smaller than 0.01 mmol/g, at a temperature above 250° C., preferably at a temperature in the range of greater than 250° C. to 650° C. It is preferred that the acid site density is determined according to Reference Example 6.

It is preferred that from 99 to 100 weight-%, more preferably from 99.5 to 100 weight-%, more preferably from 99.9 to 100 weight-%, of the molding consist of the zeolitic material and the oxidic binder.

It is preferred that the molding is a strand, preferably having a hexagonal, rectangular, quadratic, triangular, oval, or circular cross-section, more preferably a circular cross-section, wherein the cross-section has a diameter preferably in the range of from 1.5 to 3.5 mm, more preferably in the range of from 2.0 to 3.0 mm, more preferably in the range of from 2.2 to 2.8 mm, more preferably in the range of from 2.4 to 2.6 mm.

It is preferred that the molding exhibits a selectivity towards olefins in the range of from 50 to 90%, more preferably in the range of from 55 to 80%, more preferably in the range of from 60 to 75%. It is preferred that the selectivity towards olefins is determined according to Example 13.

It is preferred that the molding exhibits a selectivity towards butylenes, preferably towards one or more of but-1-ene, (2Z)-but-2-ene, (2E)-but-2-ene, 2-methylprop-1-ene, in the range of from 10 to 30%, more preferably in the range of from 15 to 25%, more preferably in the range of from 18 to 22%. It is preferred that the selectivity towards olefins is determined according to Example 13.

It is preferred that the molding exhibits a selectivity towards propylene in the range of from 20 to 100%, preferably in the range of from 25 to 90%, more preferably in the range of from 30 to 70%, preferably in the range of from 35 to 65%, more preferably in the range of from 37 to 50%, more preferably in the range of from 38 to 47%. It is preferred that the selectivity towards olefins is determined according to Example 13.

It is preferred that the molding exhibits a selectivity towards ethylene in the range of from 1 to 15%, more preferably in the range of from 4 to 12%, more preferably in the range of from 5 to 10%. It is preferred that the selectivity towards olefins is determined according to Example 13.

Yet further, the present invention relates to a method for the conversion of oxygenates to olefins comprising

(a) providing a molding according to any one of the embodiments disclosed herein;
(b) providing a gas stream comprising one or more oxygenates and optionally one or more olefins and/or optionally one or more hydrocarbons;
(c) contacting the molding provided in (a) with the gas stream provided in (b) and converting one or more oxygenates to one or more olefins and optionally to one or more hydrocarbons;
(d) optionally recycling one or more of the one or more olefins and/or of the one or more hydrocarbons contained in the gas stream obtained in (c) to (b).

It is preferred that the molding is provided in a fixed bed or in a fluidized bed.

The method may comprise further method steps, in particular with respect to an activation or regeneration of the molding. It is preferred that the method further comprises after (a) and prior to (b)

(a′) treating the molding provided in (a) with a gas stream comprising water.

It is preferred that the gas stream in (a′) has a temperature in the range of from 450 to 510° C., more preferably in the range of from 460 to 500° C., more preferably in the range of from 470 to 490° C.

It is preferred that the gas stream provided in (b) comprises one or more oxygenates selected from the group consisting of aliphatic alcohols, ethers, carbonyl compounds and mixtures of two or more thereof, more preferably from the group consisting of (C₁-C₆) alcohols, di(C₁-C₃)alkyl ethers, (C₁-C₆) aldehydes, (C₂-C₆) ketones and mixtures of two or more thereof, more preferably consisting of (C₁-C₄) alcohols, di(C₁-C₂)alkyl ethers, (C₁-C₄) aldehydes, (C₂-C₄) ketones and mixtures of two or more thereof, more preferably from the group consisting of methanol, ethanol, n-propanol, isopropanol, butanol, dimethyl ether, diethyl ether, ethyl methyl ether, diisopropyl ether, di-n-propyl ether, formaldehyde, dimethyl ketone and mixtures of two or more thereof, more preferably from the group consisting of methanol, ethanol, dimethyl ether, diethyl ether, ethyl methyl ether and mixtures of two or more thereof, the gas stream more preferably comprising methanol and/or dimethyl ether, more preferably methanol.

It is preferred that the content of oxygenates in the gas stream provided in (b) is in the range of from 2 to 100% by volume based on the total volume, more preferably from 3 to 99% by volume, more preferably from 4 to 95% by volume, more preferably from 5 to 80% by volume, more preferably from 6 to 50% by volume, more preferably from 10 to 40% by volume, more preferably from 15 to 25% by volume, more preferably from 18 to 22% by volume.

It is preferred that the gas stream provided in (b) comprises water, wherein the water content in the gas stream provided in (b) is more preferably in the range from 1 to 90% by volume, more preferably in the range of from 2 to 80% by volume, more preferably in the range of from 5 to 75% by volume, more preferably from 10 to 70% by volume.

It is preferred that the gas stream provided in (b) further comprises one or more diluting gases, more preferably one or more diluting gases in an amount in the range of from 0.1 to 90% by volume, more preferably from 1 to 85% by volume, more preferably from 5 to 80% by volume, more preferably from 10 to 75% by volume.

It is preferred that the one or more diluting gases are selected from the group consisting of H₂O, helium, neon, argon, krypton, nitrogen, carbon monoxide, carbon dioxide, and mixtures of two or more thereof, more preferably from the group consisting of H₂O, argon, nitrogen, carbon dioxide, and mixtures of two or more thereof, wherein more preferably the one or more diluting gases comprise H₂O or nitrogen, wherein more preferably the one or more diluting gases is H₂O or nitrogen.

It is preferred that contacting in (c) is effected at a temperature in the range from 225 to 700° C., preferably from 275 to 650° C., more preferably from 325 to 600° C., more preferably from 375 to 550° C., more preferably from 425 to 525° C., and more preferably from 450 to 500° C., more preferably from 475 to 495, more preferably from 480 to 490° C.

According to one alternative, it is preferred that contacting in (c) is effected at a pressure in the range from 0.01 to 25 bar, more preferably from 0.1 to 20 bar, more preferably from 0.25 to 15 bar, more preferably from 0.5 to 10 bar, more preferably from 0.75 to 5 bar, more preferably from 0.8 to 2 bar, more preferably from 0.85 to 1.5 bar, more preferably from 0.9 to 1.1 bar.

According to another alternative, it is preferred that contacting in (c) is effected at a pressure in the range from 0.1 to 25 bar(gauge), preferably from 0.25 to 20 bar(gauge), more preferably from 0.5 to 15 bar(gauge), more preferably from 1.0 to 10 bar(gauge), more preferably from 2.0 to 7.0 bar(gauge), more preferably from 3.0 to 5.0 bar(gauge), more preferably from 3.9 to 4.1 bar(gauge).

In the case where the method is a continuous method, it is preferred that the gas hourly space velocity (GHSV) of the contacting in (c) is in the range from 1 to 30,000 h⁻¹, more preferably from 1,000 to 25,000 h⁻¹, preferably from 10,000 to 23,000 h⁻¹, more preferably from 15,000 to 21,500 h⁻¹, more preferably from 20,000 to 20,500 h⁻¹.

Further in the case where the method is a continuous method, it is preferred that the weight hourly space velocity (WHSV) of the contacting in (c) is in the range from 0.5 to 50 h⁻¹, more preferably from 1 to 30 h⁻¹, more preferably from 2 to 20 h⁻¹, preferably from 5 to 15 h⁻¹, more preferably from 8 to 12 h⁻¹, more preferably from 9 to 11 h⁻¹.

It is preferred that the one or more olefins and/or one or more hydrocarbons optionally provided in (b) and/or optionally recycled to (b) comprise one or more selected from the group consisting of ethylene, (C₄-C₇)olefins, (C₄-C₇)hydrocarbons, and mixtures of two or more thereof, and preferably from the group consisting of ethylene, (C₄-C₅)olefins, (C₄-C₅)hydrocarbons, and mixtures of two or more thereof.

As disclosed above, the method may comprise further method steps. It is preferred that the method further comprises,

(e) regenerating the molding in a gas stream comprising one or more of oxygen and nitrogen, preferably air or lean air.

It is preferred that regeneration in (e) is performed in-situ.

It is preferred that the temperature of the gas stream in (e) comprising a mixture of air and nitrogen has a temperature in the range of from 450 to 550° C., more preferably in the range of from 470 to 510° C., more preferably in the range of from 480 to 500° C.

Yet further, the present invention relates to a use of a molding according to any one of the embodiments disclosed herein as a molecular sieve, as an adsorbent, for ion-exchange, or as a catalyst and/or as a catalyst support, preferably as a catalyst for the selective catalytic reduction (SCR) of nitrogen oxides NOₓ; for the oxidation of NH₃, in particular for the oxidation of NH₃ slip in diesel systems; for the decomposition of N₂O; as an additive in fluid catalytic cracking (FCC) processes; and/or as a catalyst in organic conversion reactions, preferably as a hydrocracking catalyst, as an alkylation catalyst, as an isomerization catalyst, or as a catalyst in the conversion of alcohols to olefins, and more preferably in the conversion of oxygenates to olefins.

It is preferred that the molding is used in a methanol-to-olefin process (MTO process), in a dimethylether to olefin process (DTO process), methanol-to-gasoline process (MTG process), in a methanol-to-hydrocarbon process, in a methanol to aromatics process, in a biomass to olefins and/or biomass to aromatics process, in a methane to benzene process, for alkylation of aromatics, or in a fluid catalytic cracking process (FCC process), preferably in a methanol-to-olefin process (MTO process) and/or in a dimethylether to olefin process (DTO process), and more preferably in a methanol-to-propylene process (MTP process), in a methanol-to-propylene/butylene process (MT3/4 process), in a dimethylether-to-propylene process (DTP process), in a dimethylether-to-propylene/butylene process (DT3/4 process), and/or in a dimethylether-to-ethylene/propylene (DT2/3 process).

In the context of the present invention, a weight of one or more alkaline earth metals is calculated as the weight of the respective alkaline earth metals as element or the sum the weights of the respective alkaline earth metals as elements. For example, if the one or more alkaline earth metals is Mg, the weight of said alkaline earth metals is calculated as elemental Mg. As a further example, if the one or more alkaline earth metals consists of Mg and Ba, the weight of said alkaline earth metals is calculated as elemental Mg and Ba.

In the context of the present invention, a weight of an oxidic binder, unless otherwise stated, is calculated as the weight of the respective oxidic binder as oxide or the sum the weights of the respective oxidic binders as oxides. For example, if an oxidic binder is silica, the weight of said oxidic binder is calculated as SiO₂. As a further example, if an oxidic binder consists of a mixed oxide comprising Ti and Al, the weight of said oxidic binder is calculated as sum of TiO₂ and Al₂O₃.

In the context of the present invention, the term “based on the weight of the zeolitic material”, unless otherwise stated, refers to the weight of the zeolitic material including ion-exchanged metal ions, e.g. Mg.

The unit bar(abs) refers to an absolute pressure of 10⁵ Pa.

The present invention is further illustrated by the following set of embodiments and combinations of embodiments resulting from the dependencies and back-references as indicated. In particular, it is noted that in each instance where a range of embodiments is mentioned, for example in the context of a term such as “The molding of any one of embodiments 1 to 4”, every embodiment in this range is meant to be explicitly disclosed for the skilled person, i.e. the wording of this term is to be understood by the skilled person as being synonymous to “The molding of any one of embodiments 1, 2, 3, and 4”. Further, it is explicitly noted that the following set of embodiments is not the set of claims determining the extent of protection, but represents a suitably structured part of the description directed to general and preferred aspects of the present invention.

1. A process for preparing a molding comprising a zeolitic material and one or more oxidic binders, wherein the zeolitic material comprises YO₂ and optionally X₂O₃ in its framework structure,

wherein Y is a tetravalent element and X is a trivalent element, the process comprising
(i) preparing a mixture comprising a zeolitic material, a source of an oxidic binder, a first plasticizer, and an acid;
(ii) preferably admixing water to the mixture obtained from (i);
(iii) preferably admixing a second plasticizer to the mixture obtained from, preferably in, (i) or (ii);
(iv) preferably admixing water to the mixture obtained from, preferably in, (iii);
(v) shaping of the mixture obtained from (i), (ii), (iii), or (iv), to obtain a precursor of a molding;

wherein in the mixture obtained in (i) the weight ratio of the source of the oxidic binder, calculated as the oxide, to the sum of the zeolitic material and the source of the oxidic binder, calculated as the oxide, is in the range of from 0.05:1 to 0.15:1.

2. The process of embodiment 1, wherein (i) comprises
(i.1.a) preparing a mixture comprising a zeolitic material, a source of an oxidic binder, and a first plasticizer;
(i.1.b) admixing an acid to the mixture obtained in (i.1.a).

3. The process of embodiment 1, wherein (i) comprises
(i.2.a) providing a zeolitic material;
(i.2.b) providing a mixture comprising a source of an oxidic binder, optionally water, and an acid;
(i.2.c) mixing the mixture obtained in (i.2.b) with the zeolitic material provided in (i.2.a);
(i.2.d) admixing a first plasticizer to the mixture obtained in (i.2.c),

wherein mixing according to (i.2.c) preferably comprises admixing the mixture provided in (i.2.b) to the zeolitic material provided in (i.2.a) or admixing the zeolitic material provided in (i.2.a) to the mixture provided in (i.2.b).

4. The process of embodiment 3, wherein water is comprised in the mixture obtained in (i.2.b), wherein the mixture obtained in (i.2.b) preferably exhibits a weight ratio of water to the source of the oxidic binder, calculated as source of the oxidic binder as such, in the range of from 1:1 to 10:1, more preferably in the range of from 4.0:1 to 5.0:1, more preferably in the range of from 4.50:1 to 4.70:1.

5. The process of any one of embodiments 1 to 4, wherein the mixture prepared in (i) is mixed in a kneader, Lodige mixer (German: Lödige Mischer) or mix-muller.

6. The process of any one of embodiments 1 to 5, wherein in the mixture prepared in (i) the weight ratio of the first plasticizer to the sum of the zeolitic material and the source of the oxidic binder, calculated as source of the oxidic binder as such, is in the range of from 0.01:1 to 0.1:1, preferably in the range of from 0.02:1 to 0.08:1, more preferably in the range of from 0.03:1 to 0.07:1, more preferably in the range of from 0.04:1 to 0.06:1, more preferably in the range of from 0.045:1 to 0.055:1.

7. The process of any one of embodiments 1 to 6, wherein the first plasticizer is selected from the group consisting of organic polymers, carbohydrates, graphite, plant additives, and mixtures of two or more thereof, preferably from the group consisting of polymeric vinyl compounds, polyalkylene oxides, polyacrylates, polymethacrylates, polyolefins, polyamides, polyesters, polystyrenes, polysaccharides, and mixtures of two or more thereof, wherein the first plasticizer is more preferably a polysaccharide.

8. The process of embodiment 7, wherein the polysaccharide is selected from the group consisting of celluloses, cellulose derivatives, and starches, wherein the polysaccharide preferably is one or more of methyl cellulose and carboxmethylcellulose.

9. The process of embodiment 7 or 8, wherein the polysaccharide has a bulk density in the range of from 500 to 800 g/l, preferably in the range of from 550 to 750 g/l, more preferably in the range of from 600 to 700 g/l, more preferably in the range of from 630 to 670 g/l.

10. The process of any one of embodiments 7 to 9, wherein the polysaccharide has a viscosity in the range of from 3000 to 4000 mPas, preferably in the range of from 3400 to 3600 mPas, more preferably in the range of from 3450 to 3550 mPas.

11. The process of any one of embodiments 1 to 10, wherein the weight ratio of the source of the oxidic binder, calculated as the oxide, to the sum of the zeolitic material and the source of the oxidic binder, calculated as the oxide, in the mixture obtained in (i) is in the range of from 0.06:1 to 0.14:1, preferably in the range of from 0.07:1 to 0.13:1, more preferably in the range of from 0.08:1 to 0.12:1 more preferably in the range of from 0.09:1 to 0.11.1.

12. The process of any one of embodiments 1 to 11, wherein the source of the oxidic binder is a source of one or more of silica, alumina, and silica-alumina, preferably a source of alumina.

13. The process of any one of embodiments 1 to 12, wherein the source of the oxidic binder comprises one or more of AlOOH (boehmite), Al₂O₃, Al(OH)₃, hydrotalcite, a silica sol, a colloidal silica, a wet process silica, and a dry process silica, preferably one or more of AlOOH (boehmite) and Al₂O₃, more preferably AlOOH (boehmite), wherein the source of the oxidic binder more preferably is AlOOH (boehmite).

14. The process of any one of embodiments 1 to 13, wherein Y is selected from the group consisting of Si, Sn, Ti, Zr, Ge, and a mixture of two or more thereof, preferably from the group consisting of Si, Ti, and a mixture thereof, wherein more preferably Y is Si.

15. The process of any one of embodiments 1 to 14, wherein X is selected from the group consisting of B, Al, Ga, In, and a mixture of two or more thereof, preferably from the group consisting of B, Al and a mixture thereof, wherein more preferably X is Al.

16. The process of any one of embodiments 1 to 15, wherein the zeolitic material has a molar ratio of YO₂ to X₂O₃ in the range of from 50 to 150, preferably in the range of from 75 to 125, more preferably in the range of from 90 to 120, more preferably in the range of from 95 to 115.

17. The process of any one of embodiments 1 to 16, wherein the zeoltic material has a framework structure type selected from the group consisting of ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE, BSV, CAN, CAS, CDO, CFI, CGF, CGS, CHA, -CHI, -CLO, CON, CSV, CZP, DAC, DDR, DFO, DFT, DOH, DON, EAB, EDI, EEI, EMT, EON, EPI, ERI, ESV, ETR, EUO, *-EWT, EZT, FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH,*-ITN,ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MFI, MFS, MON, MOR, MOZ, *MRE, MSE, MSO, MTF, MTN, MTT, MTW, MVY, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF, *-SSO, SSY, STF, STI, *STO, STT, STW, -SVR, SW, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, and mixed types of two or more thereof, preferably from the group consisting of MFI, MEL, ITH, IWR, CON, and mixed types of two or more thereof, more preferably selected from the group consisting of MFI, ITH, IWR, CON, and mixed types of two or more thereof, wherein the zeolitic material more preferably has the MFI framework structure type.

18. The process of any one of embodiments 1 to 17, wherein the zeolitic material has an MFI-type framework structure, wherein the zeolitic material is selected from the group consisting of Silicalite, ZSM-5, [Fe—Si—O]-MFI, [Ga—Si—O]-MFI, [As—Si—O]-MFI, AMS-1B, AZ-1, Bor-C, Encilite, Boralite C, FZ-1, LZ-105, Mutinaite, NU-4, NU-5, TS-1, TSZ, TSZ-III, TZ-01, USC-4, USI-108, ZBH, ZKQ-1B, ZMQ-TB, MnS-1, and FeS-1, including mixtures of two or more thereof, preferably from the group consisting of Silicalite, ZSM-5, AMS-1B, AZ-1, Encilite, FZ-1, LZ-105, Mutinaite, NU-4, NU-5, TS-1, TSZ, TSZ-III, TZ-01, USC-4, USI-108, ZBH, ZKQ-1B, and ZMQ-TB, including mixtures of two or more thereof, wherein more preferably the zeolitic material having an MFI-type framework structure comprises Silicalite and/or ZSM-5, preferably ZSM-5, wherein more preferably the zeolitic material having an MFI-type framework structure is zeolite Silicalite and/or ZSM-5, preferably ZSM-5.

19. The process of any one of embodiments 1 to 18, wherein the zeolitic material comprises one or more alkaline earth metals M, wherein the one or more alkaline earth metals M preferably are selected from the group consisting of Be, Mg, Ca, Sr, Ba, and a mixture of two or more thereof, preferably from the group consisting of Mg, Ca, and a mixture thereof, wherein more preferably the alkaline earth metal M comprises, more preferably is, Mg.

20. The process of embodiment 19, wherein the zeolitic material comprises the alkaline earth metal M in an amount in the range of from 0.5 to 4.0 weight-%, calculated as the element, based on the weight of the molding, preferably in the range of from 1.0 to 3.0 weight-%, more preferably in the range of from 1.5 to 2.7 weight-%, more preferably in the range of from 1.7 to 2.5 weight-%.

21. The process of embodiment 19 or 20, wherein the zeolitic material comprises the alkaline earth metal M in an amount in the range of from 0.5 to 4.0 weight-%, calculated as the element, based on the weight of the zeolitic material, calculated as zeolitic material without the alkaline earth metal M, preferably in the range of from 1.8 to 2.6 weight-%, more preferably in the range of from 2.0 to 2.4 weight-%, more preferably in the range of from 2.1 to 2.3 weight-%.

22. The process of any one of embodiments 19 to 21, wherein the zeolitic material is impregnated with the one or more alkaline earth metals M.

23. The process of embodiment 22, wherein the zeolitic material is impregnated by spray-impregnation, adhesion impregnation, incipient impregnation, or wet impregnation adhesion technique.

24. The process of any one of embodiments 1 to 23, wherein in the mixture prepared in (i) the weight ratio of the source of the oxidic binder, calculated as source of the oxidic binder as such, to the zeolitic material is in the range of from 0.05:1 to 0.25:1, preferably in the range of from 0.07:1 to 0.22:1, more preferably in the range of from 0.10:1 to 0.19:1, more preferably in the range of from 0.11:1 to 0.18:1, more preferably in the range of from 0.12:1 to 0.17:1, more preferably in the range of from 0.13:1 to 0.16:1, more preferably in the range of from 0.14:1 to 0.15:1.

25. The process of any one of embodiments 1 to 24, wherein the acid is one or more of an inorganic acid and an organic acid, wherein the organic acid preferably is one or more of formic acid, acetic acid, propionic acid, oxalic acid, and tartaric acid, more preferably formic acid, wherein the inorganic acid preferably is one or more of hydrochloric acid, nitric acid, and phosphoric acid, more preferably nitric acid, wherein the acid more preferably comprises, preferably is, one or more of formic acid and nitric acid.

26. The process of any one of embodiments 2 to 25, wherein the acid is admixed in (i.1.b) or provided for providing the mixture in (i.2.b) as an aqueous solution, preferably as an aqueous solution comprising an amount of the acid in the range of from 5 to 50 weight-%, more preferably in the range of from 10 to 40 weight-%, more preferably in the range of from 12.5 to 37.5 weight-%, more preferably in the range of from 15 to 35 weight-%, more preferably in the range of from 17.5 to 32.5 weight-%, more preferably in the range of from 20 to 30 weight-%, more preferably in the range of from 22.5 to 27.5 weight-%, more preferable in the range of from 24 to 26 weight-%, based on the total weight of the aqueous solution, wherein the acid is preferably formic acid.

27. The process of any one of embodiments 2 to 26, wherein the acid is admixed in (i.1.b) or provided for providing the mixture in (i.2.b) as an aqueous solution, preferably as an aqueous solution comprising an amount of the acid in the range of from 1 to 20 weight-%, preferably in the range of from 3 to 15 weight-%, more preferably in the range of from 5 to 13 weight-%, more preferably in the range of from 6 to 12 weight-%, more preferably in the range o from 7 to 11 weight-%, more preferable in the range of from 8 to 10 weight-%, based on the total weight of the aqueous solution, wherein the acid is preferably nitric acid.

28. The process of embodiment 26 or 27, wherein the weight ratio of the acid admixed in (i.1.b) or provided for providing the mixture in (i.2.b) to the sum of the zeolitic material and the source of the oxidic binder of the mixture prepared in (i) is in the range of from 0.05:1 to 0.15:1, preferably in the range of from 0.06:1 to 0.14:1, preferably in the range of from 0.07:1 to 0.13:1, more preferably in the range of from 0.08:1 to 0.12:1 more preferably in the range of from 0.09:1 to 0.11:1.

29. The process of any one of embodiments 2 to 28, wherein the acid is admixed in (i.1.b) or provided for providing the mixture in (i.2.b) as an aqueous solution, preferably as an aqueous solution comprising an amount of the acid in the range of from 50 to 80 weight-%, more preferably in the range of from 55 to 75 weight-%, more preferably in the range o from 60 to 70 weight-%, more preferable in the range of from 63 to 67 weight-%, based on the total weight of the aqueous solution, wherein the acid is preferably nitric acid.

30. The process of embodiment 29, wherein the weight ratio of the acid admixed in (i.1.b) or provided for providing the mixture in (i.2.b) to the sum of the zeolitic material and the source of the oxidic binder of the mixture prepared in (i) is in the range of from 0.005:1 to 0.05:1, more preferably in the range of from 0.010:1 to 0.030:1 more preferably in the range of from 0.015:1 to 0.025:1.

31. The process of any one of embodiments 1 to 30, wherein in (ii) water is admixed to the mixture obtained from, preferably in, (i), preferably de-ionized water.

32. The process of embodiment 31, wherein in the mixture in (ii) the weight ratio of water to the sum of the source of the oxidic binder and the zeolitic material is in the range of from 0.1:1 to 1.5:1, preferably in the range of from 0.2:1 to 0.8:1, more preferably in the range of from 0.3:1 to 0.7:1, preferably in the range of from 0.3:1 to 0.6:1, more preferably in the range of from 0.4:1 to 0.5:1 more preferably in the range of from 0.45:1 to 0.46:1.

33. The process of any one of embodiments 1 to 32, wherein in (iii) a second plasticizer is admixed to the mixture obtained from, preferably in, (i) or (ii), wherein the second plasticizer preferably is different to the first plasticizer.

34. The process of embodiment 33, wherein in the mixture in (iii) the weight ratio of the second plasticizer to the sum of the source of the oxidic binder and the zeolitic material is in the range of from 0.001:1 to 0.030:1, preferably in the range of from 0.005:1 to 0.015:1, preferably in the range of from 0.007:1 to 0.013:1, more preferably in the range of from 0.008:1 to 0.012:1 more preferably in the range of from 0.009:1 to 0.011:1.

35. The process of embodiment 33 or 34, wherein the second plasticizer is selected from the group consisting of organic polymers, carbohydrates, graphite, plant additives, and mixtures of two or more thereof, preferably from the group consisting of polymeric vinyl compounds, polyalkylene oxides, polyacrylates, polymethacrylates, polyolefins, polyamides, polyesters, polystyrenes, polysaccharides, and mixtures of two or more thereof, wherein the second plasticizer more preferably is a polyethylene oxide or a polysaccharide.

36. The process of embodiment 35, wherein the polysaccharide is selected from the group consisting of celluloses, cellulose derivatives, and starches, wherein the polysaccharide is preferably one or more of methyl cellulose and carboxmethylcellulose.

37. The process of any one of embodiments 1 to 36, wherein in (iv) water is admixed to the mixture obtained from, preferably in, (i), (ii), or (iii), preferably de-ionized water.

38. The process of embodiment 37, wherein in the mixture in (iv) the weight ratio of water to the sum of the source of the oxidic binder and the zeolitic material is in the range of from 0.1:1 to 1:1, preferably in the range of from 0.30:1 to 0.90:1, more preferably in the range of from 0.50:1 to 0.7:1, more preferably in the range of from 0.55:1 to 0.65:1 more preferably in the range of from 0.60:1 to 0.61:1.

39. The process of any one of embodiments 1 to 38, wherein in (v), the mixture is shaped to a strand, preferably having a hexagonal, rectangular, quadratic, triangular, oval, or circular cross-section, more preferably a circular cross-section.

40. The process of embodiment 39, wherein the strand having a circular cross-section has a diameter in the range of from 0.5 to 7 mm, preferably in the range of from 1.5 to 3.5 mm, more preferably in the range of from 2.1 to 2.9 mm, more preferably in the range of from 2.3 to 2.7 mm, more preferably in the range of from 2.4 to 2.6 mm.

41. The process of any one of embodiments 1 to 40, wherein in (v), shaping comprises extruding the mixture.

42. The process of any one of embodiments 1 to 41, wherein shaping according to (v) further comprises drying the precursor of the molding in a gas atmosphere.

43. The process of embodiment 42, wherein drying is carried out at a temperature of the gas atmosphere in the range of from 80 to 160° C., preferably in the range of from 100 to 140° C., more preferably in the range of from 110 to 130° C.

44. The process of embodiments 42 or 43, wherein the gas atmosphere comprises nitrogen, oxygen, or a mixture thereof, wherein the gas atmosphere is preferably oxygen, air, or lean air.

45. The process of any one of embodiments 1 to 44, preferably any one of embodiment 42 to 44, wherein shaping according to (v) further comprises calcining the, preferably dried, precursor of the molding in a gas atmosphere.

46. The process of embodiment 45, wherein calcining is carried out at a temperature of the gas atmosphere in the range of from 500 to 650° C., preferably in the range of from 530 to 570° C., more preferably in the range of from 540 to 560° C.

47. The process of embodiment 45 or 46, wherein the gas atmosphere comprises nitrogen, oxygen, or a mixture thereof, wherein the gas atmosphere is preferably oxygen, air, or lean air.

48. A molding obtainable or obtained by the process of any one of embodiments 1 to 47.

49. A molding, preferably prepared according to the process of any one of embodiments 1 to 48, comprising one or more oxidic binders and a zeolitic material wherein the zeolitic material comprises YO₂ and optionally X₂O₃ in its framework structure,

wherein Y is a tetravalent element and X is a trivalent element,
wherein the molding comprises the one or more oxidic binders, calculated as the oxide, in an amount in the range of from 5 to 15 weight-%, and wherein the molding exhibits a crush strength of equal or greater than 9 N, determined according to Reference Example 5.

50. The molding of embodiment 49, exhibiting a crush strength of equal or greater than 10 N, preferably of equal or greater than 15 N, preferably equal or greater than 18 N, more preferably equal or greater than 19 N, more preferably equal or greater than 20 N, wherein the molding more preferably exhibits a crush strength in the range of from 15 to 50 N, more preferably in the range of from 17 to 30 N, determined according to Reference Example 5.

51. The molding of embodiment 49 or 50, exhibiting a diffusion coefficient in the range of from 0.40 to 1.30×10⁻⁹ m²/s, preferably in the range of from 0.60 to 1.10×10⁻⁹ m²/s, more preferably in the range of from 0.72 to 0.98×10⁻⁹ m²/s, preferably determined according to Reference Example 4.

52. The molding of any one of embodiments 49 to 51, exhibiting a tortuosity parameter relative to water in the range of from 1.00 to 3.75, preferably in the range of from 1.2 to 3.0, more preferably in the range of from 1.4 to 2.8, preferably determined as described in Reference Example 2.

53. The molding of embodiment 52, wherein the one or more oxidic binders are selected from the group consisting of silica, alumina, silica-alumina, and a mixture of two or more thereof, wherein the one or more oxidic binders preferably are alumina.

54. The molding of any one of embodiments 49 to 53, wherein the molding comprises the one or more oxidic binders, calculated as the oxide, in an amount in the range of from 6 to 14 weight-%, more preferably in the range of from 7 to 13 weight-%, more preferably in the range of from 8 to 12 weight-%, more preferably in the range of from 9 to 11 weight-%.

55. The molding of any one of embodiments 49 to 54, wherein Y is selected from the group consisting of Si, Sn, Ti, Zr, Ge, and a mixture of two or more thereof, preferably from the group consisting of Si, Ti, and a mixture thereof, wherein more preferably Y is Si.

56. The molding of any one of embodiments 49 to 55, wherein X is selected from the group consisting of B, Al, Ga, In, and a mixture of two or more thereof, preferably from the group consisting of B, Al and a mixture thereof, wherein more preferably X is Al.

57. The molding of any one of embodiments 49 to 56, wherein the zeolitic material has a framework structure type selected from the group consisting of ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE, BSV, CAN, CAS, CDO, CFI, CGF, CGS, CHA, -CHI, -CLO, CON, CSV, CZP, DAC, DDR, DFO, DFT, DOH, DON, EAB, EDI, EEI, EMT, EON, EPI, ERI, ESV, ETR, EUO, *-EWT, EZT, FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH, *-ITN, ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MFI, MFS, MON, MOR, MOZ, *MRE, MSE, MSO, MTF, MTN, MTT, MTW, MVY, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF, *-SSO, SSY, STF, STI,*STO, STT, STW, -SVR, SW, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, and mixed types of two or more thereof, preferably from the group consisting of MFI, MEL, ITH, IWR, CON, and mixed types of two or more thereof, more preferably selected from the group consisting of MFI, ITH, IWR, CON, and mixed types of two or more thereof, wherein the zeolitic material more preferably has the framework structure type MFI.

58. The molding of any one of embodiments 49 to 57, wherein the zeolitic material comprises X₂O₃, and wherein the zeolitic material has a molar ratio of YO₂ to X₂O₃ in the range of from 50 to 150, preferably in the range of from 75 to 125, more preferably in the range of from 90 to 120, more preferably in the range of from 95 to 115.

59. The molding of any one of embodiments 49 to 58, wherein the zeolitic material has the framework structure type MFI, wherein the zeolitic material having the MFI framework structure type preferably comprises one or more of ZSM-5, ZBM-10, [As—Si—O]-MFI, [Fe—Si—O]-MFI, [Ga—Si—O]-MFI, AMS-1B, AZ-1, boron-C, boralite C, encilite, FZ-1, LZ-105, monoclinic H-ZSM-5, mutinaite, NU-4, NU-5, silicalite, TS-1, TSZ, TSZ-III, TZ-01, USC-4, USI-108, ZBH, ZKQ-1B, and ZMQ-TB, preferably one or more of ZSM-5, and ZBM-10, more preferably ZSM-5.

60. The molding of any one of embodiments 49 to 59, wherein from 99 to 100 weight-%, more preferably from 99.5 to 100 weight-%, more preferably from 99.9 to 100 weight-%, of the zeolitic material consist of Y, optionally X, O, and H.

61. The molding of any one of embodiments 49 to 60, wherein the zeolitic material comprises one or more alkaline earth metals M.

62. The molding of embodiment 61, wherein the one or more alkaline earth metals M are selected from the group consisting of Be, Mg, Ca, Sr, Ba, and a mixture of two or more thereof, preferably from the group consisting of Mg, Ca, and a mixture thereof, wherein more preferably the one or more alkaline earth metals M comprise, more preferably consist of, Mg.

63. The molding of embodiment 61 or 62, wherein the zeolitic material comprises the one or more alkaline earth metals M in an amount in the range of from 0.1 to 5 weight-%, calculated as element, based on the weight of the molding, preferably in the range of from 1.5 to 2.5 weight-%, more preferably in the range of from 1.7 to 2.3 weight-%.

64. The molding of embodiment 61 or 62, wherein the zeolitic material comprises the one or more alkaline earth metals M in an amount in the range of from 0.5 to 4.0 weight-%, calculated as the element, based on the weight of the zeolitic material, preferably in the range of from 1.0 to 3.0 weight-%, more preferably in the range of from 1.5 to 2.7 weight-%, more preferably in the range of from 1.7 to 2.5 weight-%, more preferably in the range of from 1.7 to 2.3 weight-%.

65. The molding of any one of embodiments 61 to 64, wherein the zeolitic material is impregnated, preferably spray-impregnated, with the one or more alkaline earth metals M.

66. The molding of any one of embodiments 61 to 65, wherein from 99 to 100 weight-%, more preferably from 99.5 to 100 weight-%, more preferably from 99.9 to 100 weight-%, of the zeolitic material consist of Y, optionally X, O, H, and the one or more alkaline earth metals M.

67. The molding of any one of embodiments 49 to 66, comprising less than 1 weight-% of sodium, preferably less than 0.1 weight-% of sodium, more preferably less than 0.01 weight-% of sodium.

68. The molding of any one of embodiments 49 to 67, having a BET specific surface area in the range of from 300 to 400 m²/g, preferably in the range of from 325 to 375 m²/g, more preferably in the range of from 350 to 360 m²/g, preferably determined as described in Reference Example 1.

69. The molding of any one of embodiments 49 to 68, having a total pore volume in the range of from 0.2 to 0.75 g/ml, preferably in the range of from 0.45 to 0.53 g/ml, more preferably in the range of from 0.47 to 0.51 g/ml, more preferably in the range of from 0.48 to 0.50 g/ml, determined according to Reference Example 3.

70. The molding of any one of embodiments 49 to 69, exhibiting an acid site density in the range of from 0.20 to 0.75 mmol/g, preferably in the range of from 0.25 to 0.65 mmol/g, more preferably in the range of from 0.44 to 0.52 mmol/g, more preferably in the range of from 0.46 to 0.50 mmol/g, more preferably in the range of from 0.47 to 0.49 mmol/g, at a temperature below 250 ° C., determined according to Reference Example 6.

71. The molding of any one of embodiments 49 to 70, exhibiting an acid site density of equal or smaller than 0.5 mmol/g, preferably of equal or smaller than 0.30 mmol/g, more preferably of equal or smaller than 0.25 mmol/g, more preferably of equal or smaller than 0.1 mmol/g, more preferably of equal or smaller than 0.01 mmol/g, at a temperature above 250° C., preferably at a temperature in the range of greater than 250° C. to 650° C., determined according to Reference Example 6.

72. The molding of any one of embodiments 49 to 71, wherein from 99 to 100 weight-%, more preferably from 99.5 to 100 weight-%, more preferably from 99.9 to 100 weight-%, of the molding consist of the zeolitic material and the oxidic binder.

73. The molding of any one of embodiments 49 to 72, being a strand, preferably having a hexagonal, rectangular, quadratic, triangular, oval, or circular cross-section, more preferably a circular cross-section, wherein the cross-section has a diameter preferably in the range of from 1.5 to 3.5 mm, more preferably in the range of from 2.0 to 3.0 mm, more preferably in the range of from 2.2 to 2.8 mm, more preferably in the range of from 2.4 to 2.6 mm.

74. The molding of any one of embodiments 49 to 73, exhibiting a selectivity towards olefins in the range of from 50 to 90%, preferably in the range of from 55 to 80%, more preferably in the range of from 60 to 75%, preferably determined according to Example 13.

75. The molding of any one of embodiments 49 to 74, exhibiting a selectivity towards butylenes, preferably towards one or more of but-1-ene, (2Z)-but-2-ene, (2E)-but-2-ene, 2-methylprop-1-ene, in the range of from 10 to 30%, preferably in the range of from 15 to 25%, more preferably in the range of from 18 to 22%, preferably determined according to Example 13.

76. The molding of any one of embodiments 49 to 75, exhibiting a selectivity towards propylene in the range of from 20 to 100%, preferably in the range of from 25 to 90%, more preferably in the range of from 30 to 70%, preferably in the range of from 35 to 65%, more preferably in the range of from 37 to 50%, more preferably in the range of from 38 to 47%, preferably determined according to Example 13.

77. The molding of any one of embodiments 49 to 76, exhibiting a selectivity towards ethylene in the range of from 1 to 15%, preferably in the range of from 4 to 12%, more preferably in the range of from 5 to 10%, preferably determined according to Example 13.

78. A method for the conversion of oxygenates to olefins comprising

(a) providing a molding according to any one of embodiments 48 to 77;
(b) providing a gas stream comprising one or more oxygenates and optionally one or more olefins and/or optionally one or more hydrocarbons;
(c) contacting the molding provided in (a) with the gas stream provided in (b) and converting one or more oxygenates to one or more olefins and optionally to one or more hy-drocarbons;
(d) optionally recycling one or more of the one or more olefins and/or of the one or more hydrocarbons contained in the gas stream obtained in (c) to (b).

79. The method of embodiment 78, wherein the molding is provided in a fixed bed or in a fluidized bed.

80. The method of embodiment 78 or 79, wherein the method further comprises after (a) and prior to (b)

(a′) treating the molding provided in (a) with a gas stream comprising water.

81. The method of embodiment 80, wherein the gas stream has a temperature in the range of from 450 to 510° C., preferably in the range of from 460 to 500° C., more preferably in the range of from 470 to 490° C.

82. The method of any one of embodiments 78 to 81, wherein the gas stream provided in (b) comprises one or more oxygenates selected from the group consisting of aliphatic alcohols, ethers, carbonyl compounds and mixtures of two or more thereof, preferably from the group consisting of (C₁-C₆) alcohols, di(C₁-C₃)alkyl ethers, (C₁-C₆) aldehydes, (C₂-C₆) ketones and mixtures of two or more thereof, more preferably consisting of (C₁-C₄) alcohols, di(C₁-C₂)alkyl ethers, (C₁-C₄) aldehydes, (C₂-C₄) ketones and mixtures of two or more thereof, more preferably from the group consisting of methanol, ethanol, n-propanol, isopropanol, butanol, dimethyl ether, diethyl ether, ethyl methyl ether, diisopropyl ether, di-n-propyl ether, formaldehyde, dimethyl ketone and mixtures of two or more thereof, more preferably from the group consisting of methanol, ethanol, dimethyl ether, diethyl ether, ethyl methyl ether and mixtures of two or more thereof, the gas stream more preferably comprising methanol and/or dimethyl ether, more preferably methanol.

83. The method of any one of embodiments 78 to 82, wherein the content of oxygenates in the gas stream provided in (b) is in the range of from 2 to 100% by volume based on the total volume, preferably from 3 to 99% by volume, more preferably from 4 to 95% by volume, more preferably from 5 to 80% by volume, more preferably from 6 to 50% by volume, more preferably from 10 to 40% by volume, more preferably from 15 to 25% by volume, more preferably from 18 to 22% by volume.

84. The method of any one of embodiments 78 to 83, wherein the gas stream provided in (b) comprises water, wherein the water content in the gas stream provided in (b) is preferably in the range from 1 to 90% by volume, more preferably in the range of from 2 to 80% by volume, more preferably in the range of from 5 to 75% by volume, more preferably from 10 to 70% by volume.

85. The method of any one of embodiments 78 to 84, wherein the gas stream provided in (b) further comprises one or more diluting gases, preferably one or more diluting gases in an amount in the range of from 0.1 to 90% by volume, more preferably in the range of from 1 to 85% by volume, more preferably in the range of from 5 to 80% by volume, more preferably in the range of from 10 to 75% by volume.

86. The method of embodiment 85, wherein the one or more diluting gases are selected from the group consisting of H₂O, helium, neon, argon, krypton, nitrogen, carbon monoxide, carbon dioxide, and mixtures of two or more thereof, preferably from the group consisting of H₂O, argon, nitrogen, carbon dioxide, and mixtures of two or more thereof, wherein more preferably the one or more diluting gases comprise H₂O or nitrogen, wherein more preferably the one or more diluting gases is H₂O or nitrogen.

87. The method of any one of embodiments 78 to 86, wherein contacting in (c) is effected at a temperature in the range from 225 to 700° C., preferably from 275 to 650° C., more preferably from 325 to 600° C., more preferably from 375 to 550° C., more preferably from 425 to 525° C., and more preferably from 450 to 500° C., more preferably from 475 to 495, more preferably from 480 to 490° C.

88. The method of any one of embodiments 78 to 87, wherein contacting in (c) is effected at a pressure in the range from 0.01 to 25 bar, preferably from 0.1 to 20 bar, more preferably from 0.25 to 15 bar, more preferably from 0.5 to 10 bar, more preferably from 0.75 to 5 bar, more preferably from 0.8 to 2 bar, more preferably from 0.85 to 1.5 bar, more preferably from 0.9 to 1.1 bar.

89. The method of any one of embodiments 78 to 87, wherein contacting in (c) is effected at a pressure in the range of from 0.1 to 25 bar(gauge), preferably from 0.25 to 20 bar(gauge), more preferably from 0.5 to 15 bar(gauge), more preferably from 1.0 to 10 bar(gauge), more preferably from 2.0 to 7.0 bar(gauge), more preferably from 3.0 to 5.0 bar(gauge), more preferably from 3.9 to 4.1 bar(gauge).

90. The method of any one of embodiments 78 to 89, wherein the method is a continuous method, wherein the gas hourly space velocity (GHSV) of the contacting in (c) is preferably in the range from 1 to 30,000 h⁻¹, preferably from 1,000 to 25,000 h⁻¹, preferably from 10,000 to 23,000 h⁻¹, more preferably from 15,000 to 21,500 h⁻¹, more preferably from 20,000 to 20,500 h⁻¹.

91. The method of any one of embodiments 78 to 90, wherein the method is a continuous method, wherein the weight hourly space velocity (WHSV) of the contacting in (c) is preferably in the range from 0.5 to 50 h⁻¹, preferably from 1 to 30 h⁻¹, more preferably from 2 to 20 h⁻¹, preferably from 5 to 15 h⁻¹, more preferably from 8 to 12 h⁻¹, more preferably from 9 to 11 h⁻¹.

92. The method of any one of embodiments 78 to 91, wherein the one or more olefins and/or one or more hydrocarbons optionally provided in (b) and/or optionally recycled to (b) comprise one or more selected from the group consisting of ethylene, (C₄-C₇)olefins, (C₄-C₇)hydrocarbons, and mixtures of two or more thereof, and preferably from the group consisting of ethylene, (C₄-C₅)olefins, (C₄-C₅)hydrocarbons, and mixtures of two or more thereof.

93. The method of any one of embodiments 78 to 92, wherein the method further comprises,

(e) regenerating the molding in a gas stream comprising one or more of oxygen and nitrogen, preferably air or lean air.

94. The method of embodiment 93, wherein the regeneration in (e) is performed in-situ.

95. The method of embodiment 93 or 94, wherein the temperature of the gas stream comprising a mixture of air and nitrogen has a temperature in the range of from 450 to 550° C., preferably in the range of from 470 to 510° C., more preferably in the range of from 480 to 500° C.

96. Use of a molding according to any one of embodiments 48 to 77 as a molecular sieve, as an adsorbent, for ion-exchange, or as a catalyst and/or as a catalyst support, preferably as a catalyst for the selective catalytic reduction (SCR) of nitrogen oxides NOₓ; for the oxidation of NH₃, in particular for the oxidation of NH₃ slip in diesel systems; for the decomposition of N₂O; as an additive in fluid catalytic cracking (FCC) processes; and/or as a catalyst in organic conversion reactions, preferably as a hydrocracking catalyst, as an alkylation catalyst, as an isomerization catalyst, or as a catalyst in the conversion of alcohols to olefins, and more preferably in the conversion of oxygenates to olefins.

97. The use of embodiment 96, wherein the molding is used in a methanol-to-olefin process (MTO process), in a dimethylether to olefin process (DTO process), methanol-to-gasoline process (MTG process), in a methanol-to-hydrocarbon process, in a methanol to aromatics process, in a biomass to olefins and/or biomass to aromatics process, in a methane to benzene process, for alkylation of aromatics, or in a fluid catalytic cracking process (FCC process), preferably in a methanol-to-olefin process (MTO process) and/or in a dimethylether to olefin process (DTO process), and more preferably in a methanol-to-propylene process (MTP process), in a methanol-to-propylene/butylene process (MT3/4 process), in a dimethylether-to-propylene process (DTP process), in a dimethylether-to-propylene/butylene process (DT3/4 process), and/or in a dimethylether-to-ethylene/propylene (DT2/3 process).

The present invention is further illustrated by the following examples and reference examples.

### EXPERIMENTAL SECTION

### Reference Example 1: Determination of the BET Specific Surface Area and Langmuir Specific Surface Area

The BET specific surface area and the Langmuir specific surface area were determined via nitrogen physisorption at 77 K according to the method disclosed in DIN 66131. The N₂ sorption isotherms at the temperature of liquid nitrogen were measured using Micrometrics ASAP 2020M and Tristar system for determining the BET specific surface area.

### Reference Example 2: Determination of the Tortuosity Parameter Relative to Water

PFG NMR enables the destruction free examination of thermal molecular motion, in free gases and liquids, in macro and supra molecular solutions and of adsorbed molecules in porous systems. The principle and applications are as described in US 20070099299 A1. From the diffusion coefficient obtained by NMR according to Reference Example 4, the tortuosity factor was calculated. The tortuosity factor of a porous material is determined from the self diffusion coefficient of a probe molecule in the porous system (D_{eff}) and the self diffusion coefficient of the free liquid (D₀) according to formula I (see S. Kolitcheff, E. Jolimaitre, A. Hugon, J. Verstraete, M. Rivallan, P-L. Carrette, F. Couenne and M. Tayakout-Fayolle, Catal. Sci. Technol., 2018, 8, 4537; and F. Elwinger, P. Pourmand, and I. Furo, J. Phys. Chem. C. 2017, 121, 13757-13764):

$$τ = \frac{{D}_{0}}{{D}_{eff}} . \qquad ( I )$$

The free diffusion coefficient for water was taken as 2.02×10⁻⁹ m² s⁻¹ at 20° C. (see M. Holz, S. R. Heil and A. Sacco. Phys. Chem. Chem. Phys., 2000, 2, 4740-4742).

### Reference Example 3: Determination of the Total Pore Volume

The total pore volume was determined via intrusion mercury porosimetry according to DIN 66133.

### Reference Example 4: Determination of the Diffusion Coefficient by NMR

Samples were prepared for NMR analyses by drying a small quantity (0.05-0.2 g) of catalyst at T>350° C. under vacuum overnight in NMR measurement tubes. The sample was then filled via a vacuum line with nanopure water (Millipore Advantage A10) to 90% of the pore volume of the catalyst support (determined by Hg-porosimetry). The filled sample was then flame sealed into the measurement tube and left overnight before measurement.

The NMR analyses to determine D_{eff} for water in the catalyst materials were conducted at 20° C. and 1 bar at 400 MHz 1H resonance frequency with Bruker Avance III NMR spectrometer. A Bruker Diff50 probe head was used with Bruker Great 60A gradient amplifiers. A temperature of 20° C. was maintained with water cooled gradient coils. The pulse program used for the PFG NMR self-diffusion analyses was the stimulated spin echo with pulsed field gradients according to FIG. 1b of US 20070099299 A1. For each sample, the spin echo attenuation curves were measured at different diffusion times (between 20 and 100 ms) by stepwise increase in the intensity of the field gradients (to a maximum gmax=3 T/m). The gradient pulse length was 1 ms. Spin echo attenuation curves were fitted to equation 6 of US 2007/0099299 A, by way of an example, a double logarithmic plot of data from a catalyst support at the various diffusion times used is shown in figure X. The slope of each line corresponds to a diffusion coefficient. The average diffusion coefficient, across all diffusion times, was used to calculate tortuosity for each catalyst support, according to Formula I (see Reference Example 2).

### Reference Example 5: Determination of the Crush Strength

The crush strength as referred to in the context of the present invention is to be understood as having been determined via a crush strength test machine Z2.5/TS1S, supplier Zwick GmbH & Co., D-89079 Ulm, Germany. As to fundamentals of this machine and its operation, reference is made to the respective instructions handbook “Register 1: Betriebsanleitung/Sicherheit-shandbuch für die Material-Prüfmaschine Z2.5/TS1S”, version 1.5, December 2001 by Zwick GmbH & Co. Technische Dokumentation, August-Nagel-Strasse 11, D-89079 Ulm, Germany. The machine was equipped with a fixed horizontal table on which the strand was positioned. A plunger having a diameter of 3 mm which was freely movable in vertical direction actuated the strand against the fixed table. The apparatus was operated with a preliminary force of 0.5 N, a shear rate under preliminary force of 10 mm/min and a subsequent testing rate of 1.6 mm/min. The vertically movable plunger was connected to a load cell for force pick-up and, during the measurement, moved toward the fixed turntable on which the molding (strand) to be investigated is positioned, thus actuating the strand against the table. The plunger was applied to the strands perpendicularly to their longitudinal axis. With said machine, a given strand as described below was subjected to an increasing force via a plunger until the strand was crushed. The force at which the strand crushes is referred to as the crushing strength of the strand.

Controlling the experiment was carried out by means of a computer which registered and evaluated the results of the measurements. The values obtained are the mean value of the measurements for 20 or 30 strands in each case. In particular, for Comparative Example 9 and Example 10 twenty strands were used and for Examples 11 and 12 thirty strands were used for the determination of the crush strength.

### Reference Example 6: Temperature Programmed Desorption of Ammonia (NH₃-TPD)

The temperature-programmed desorption of ammonia (NH₃-TPD) was conducted in an automated chemisorption analysis unit (Micromeritics AutoChem II 2920) having a thermal conductivity detector. Continuous analysis of the desorbed species was accomplished using an online mass spectrometer (OmniStar QMG200 from Pfeiffer Vacuum). The sample (0.1 g) was introduced into a quartz tube and analysed using the program described below. The temperature was measured by means of a Ni/Cr/Ni thermocouple immediately above the sample in the quartz tube. For the analyses, He of purity 5.0 was used. Before any measurement, a blank sample was analysed for calibration.

1. Preparation: Commencement of recording; one measurement per second. Wait for 10 minutes at 25° C. and a He flow rate of 30 cm³/min (room temperature (about 25° C.) and 1 atm); heat up to 600° C. at a heating rate of 20 K/min; hold for 10 minutes. Cool down under a He flow (30 cm³/min) to 100° C. at a cooling rate of 20 K/min (furnace ramp temperature); Cool down under a He flow (30 cm³/min) to 100° C. at a cooling rate of 3 K/min (sample ramp temperature).

2. Saturation with NH₃: Commencement of recording; one measurement per second. Change the gas flow to a mixture of 10% NH₃ in He (75 cm³/min; 100° C. and 1 atm) at 100° C.; hold for 30 minutes.

3. Removal of the excess: Commencement of recording; one measurement per second. Change the gas flow to a He flow of 75 cm³/min (100° C. and 1 atm) at 100° C.; hold for 60 min.

4. NH₃-TPD: Commencement of recording; one measurement per second. Heat up under a He flow (flow rate: 30 cm³/min) to 600° C. at a heating rate of 10 K/min; hold for 30 minutes.

5. End of measurement.

Desorbed ammonia was measured by means of the online mass spectrometer, which demonstrates that the signal from the thermal conductivity detector was caused by desorbed ammonia. This involved utilizing the m/z=16 signal from ammonia in order to monitor the desorption of the ammonia. The amount of ammonia adsorbed (mmol/g of sample) was ascertained by means of the Micromeritics software through integration of the TPD signal with a horizontal baseline.

### Reference Example 7: Synthesis of ZSM-5 Zeolite Having a SiO₂:Al₂O₃ Molar Ratio of 100

757.0 kg Tetraethylorthosilicate (TEOS) was stirred in a vessel. A mixture of 350 kg deionized water and 366.0 kg of an aqueous solution of tetrapropylammonium hydroxide in water (TPAOH; Sachem; 40 weight-% TPAOH in water) were admixed. The resulting mixture was stirred for 60 minutes. Then, 120 kg of deionized water were admixed. The resulting mixture is stirred for 1 h allowing for TEOS hydrolysis. The mixture was heated to an internal temperature of 90° C. whereby the external temperature was 120° C. The ethanol was removed as an azeotrope mixture of water and ethanol via distillation until a sump temperature of 95° C. was reached. Thereby, 856 kg of water/ethanol were removed from the mixture. Then, the mixture was cooled to 30° C. Then, 856 kg of water were admixed to replace the lost liquid. A solution of 24.2 kg aluminum sulfate octadecahydrate (Al₂(SO₄)₃.18 H₂O;Sigma-Aldrich) and 40 kg deionized water were admixed to the mixture. The vessel was closed and heated to a temperature of 170° C. within 4 h. The mixture was heated at 170° C. for 48 h in an autoclave. Then, the mixture was cooled to a temperature of 50° C. The mixture was treated with 177.5 kg aqueous nitric acid (BASF; 10 weight-% in water) until a pH value of 7.6 was reached. After stirring for 30 minutes, the resulting suspension was filtered. The filter cake was washed with deionized water, pre dried under a stream of nitrogen for 6 h, and subsequently dried for 36 h at a temperature of 120° C. in a dryer. 217 kg of dried material were obtained. The dry powder was ground and subsequently calcined (5 h, 500° C.).

The resulting material had a silica-to-alumina ratio of 100 and a crystallinity of higher than 90%. It displayed a BET specific surface area of 427 m²/g and a Langmuir specific surface area of 589 m²/g. Further, the resulting material had a TOC of less than 0.1 g/100 g, a Si content of 44 g/100 g, an Al content of 0.87 g/100 g, and an alkali metal content of less than 0.01 g/100 g.

### Reference Example 8: Preparation of a Zeolitic Material Comprising Mg (Mg-ZSM-5)

The ZSM-5 powder obtained from Reference Example 7 was spray impregnated with a magnesium nitrate solution. The amount of Mg weighed in was such that the powder after the calcination comprised 2-3 weight-% Mg.

For the impregnation of the ZSM-5 zeolite prepared according to Reference Example 7, 5 kg of zeolite powder were introduced into a tumble mixer. 1.2 kg of magnesium nitrate hexahydrate (Merck) were dissolved in 1.16 kg of deionized water. The resulting magnesium nitrate solution was then sprayed onto the ZSM-5 powder through a glass spray nozzle while rotating over a time period of 95 min. The mixture was subsequently rotated for further 15 minutes. Then, the impregnated powder was dried at a temperature of 120° C. for 4 h in a circulation oven, then calcined at 500° C. in a static oven for 5 h under air (heating rate of static oven was 2° C./min).

The resulting material contained 2.2 g Mg/100 g.

### Comparative Example 9: Preparation of an Extrudate Comprising Mg-ZSM-5

The Mg-ZSM-5 powder prepared according to Reference Example 8 by spray impregnation was further processed with boehmite (Pural SB; Sasol) as a binder to give extrudates. The amounts of starting materials were chosen such that the extrudates contain 10 weight-% Al₂O₃ as binder.

4970 g of zeolite powder and 790 g of boehmite (Pural SB; Sasol) were weighed in a koller and mixed for 5 minutes. 124 g aqueous formic acid (24 g formic acid in 100 g deionized water) were admixed thereto. Four portions of each 455 g of water were subsequently admixed in intervals of about 5 min over a the first 35 minutes. Then, 110 g of polyethylene oxide (PEO E160) were admixed and subsequently four portions of each 455 g of water were subsequently admixed in intervals of about 5 min until a total kneading time of 50 minutes was achieved. The kneaded material was pressed with the aid of an extrudate press through a 2.5 mm die at 120 to 200 bar. Subsequently, the resulting extrudates were dried in a circulating oven at a temperature of 120° C. for 4 h and then calcined in a static oven at 550° C. for 5 h. The extrudates may be broken manually to a desired length.

The resulting extrudates had a crush strength of 5.5 N. The Mg content of the resulting extrudates was 2.0 g Mg/100 g and the BET specific surface area was 345 m²/g. Further, the resulting extrudates displayed a total pore volume of 0.52 g/ml. In addition, the acid site density was determined according to NH₃-TPD as disclosed herein as being 0.70 mmol/g at a temperature of below 250° C. and of 0.05 mmol/g at a temperature above 250° C.

### Example 10: Preparation of an Extrudate Comprising Mg-ZSM-5 (SiO₂:Al₂O₃ Molar Ratio of 100

The Mg-ZSM-5 powder prepared according to Reference Example 8 by spray impregnation was further processed with boehmite (Pural SB; Sasol) as a binder to give extrudates. The amounts of starting materials were chosen such that the extrudates contain 10 weight-% Al₂O₃ as binder.

4900 g of zeolite powder, 726 g of boehmite (Pural SB; Sasol) and 281 g of a polysaccharide (Zusoplast PS1) were weighed in a koller and mixed for 5 minutes. 2252 g aqueous formic acid (25 weight-% of formic acid in deionized water) were admixed thereto. Four portions of each 455 g of water were subsequently admixed in intervals of about 5 min over the first 35 minutes. Then, 110 g of polyethylene oxide (PEO E160) were admixed and subsequently four portions of each 455 g of water were subsequently admixed in intervals of about 5 min until a total kneading time of 50 minutes was achieved. The kneaded material was pressed with the aid of an extrudate press through a 2.5 mm die at 120 to 200 bar. Subsequently, the resulting extrudates were dried in a circulating oven at a temperature of 120° C. for 4 h and then calcined in a static oven at 550° C. for 5 h. The extrudates may be broken manually to a desired length.

The resulting extrudates had a crush strength of 21 N. The Mg content of the resulting extrudates was 1.9 g Mg/100 g and the BET specific surface area was 356 m²/g. Further, the resulting extrudates displayed a total pore volume of 0.49 g/ml. In addition, the acid site density was determined according to NH₃-TPD as disclosed herein as being 0.48 mmol/g at a temperature of below 250° C. and of smaller than 0.01 mmol/g at a temperature above 250° C.

It can be gathered from the results for the determination of the mechanical strength that the novel molding prepared according to the present invention shows a comparatively higher crush strength than a molding prepared according to the prior art while having a similar composition. In particular, it has been shown that a molding prepared according to Example 10 of the present invention shows a crush strength of 21 N whereas a molding in accordance with the prior art shows a crush strength of 5.5 N.

### Example 11: Preparation of an Extrudate Comprising Mg-ZSM-5 (SiO₂:Al₂O₃ Molar Ratio of 100

The Mg-ZSM-5 powder prepared according to Reference Example 8 by spray impregnation was further processed with boehmite (Pural SB; Sasol) as a binder to give extrudates. The amounts of starting materials were chosen such that the extrudates contain 10 weight-% Al₂O₃ as binder.

120 g of zeolite powder were weighed in a kneader and kneaded for 5 minutes. Separately, a suspension was prepared of 17.78 g of boehmite (Pural SB; Sasol) in 80 g of deionized water. To that suspension, 4.24 g aqueous nitric acid (65 weight-% of nitric acid in deionized water) were admixed and the resulting suspension was stirred for 1 minute such that a gel was formed. Then, the formed gel was added to the zeolitic material in the kneader and the resulting mixture was kneaded for 30 minutes. 2.76 g of a polysaccharide (Zusoplast PS1) and 0.69 g of polyethylene oxide (PEO E160) were added and the resulting mixture kneaded for 5 minutes. A portion of 10 g of water was subsequently admixed and the resulting mixture was kneaded for 5 minutes. The kneaded material was pressed with the aid of an extrudate press through a 2.5 mm die at a pressure of 93 to 148 bar. Subsequently, the resulting extrudates were dried in a circulating oven at a temperature of 120° C. for 4 h (whereby the heating ramp was set to 2° C./min) and then calcined in a static oven at 550° C. for 5 h (whereby the heating ramp was set to 2° C./min). The extrudates may be broken manually to a desired length.

The resulting extrudates had a crush strength of 17.7 N. The Mg content of the resulting extrudates was 1.8 g Mg/100 g, the Al content was 5.8 g/100 g, the Si content was 38 g/100 g, the C content was less than 0.1 g/100 g, and the BET specific surface area was 350 m²/g. Further, the resulting extrudates displayed a total pore volume of 0.50 g/ml. In addition, the acid site density was determined according to NH₃-TPD as disclosed herein as being 0.325 mmol/g at a temperature of below 250° C. and 0.218 mmol/g at a temperature in the range of from greater than 250° C. to 650° C.

It can be gathered from the results for the determination of the mechanical strength that the novel molding prepared according to the present invention shows a comparatively higher crush strength than a molding prepared according to the prior art while having a similar composition. In particular, it has been shown that a molding prepared according to Example 11 of the present invention shows a crush strength of 17.7 N whereas a molding in accordance with the prior art shows a crush strength of 5.5 N.

### Example 12: Preparation of an Extrudate Comprising Mg-ZSM-5 (SiO₂:Al₂O₃ Molar Ratio of 100

The Mg-ZSM-5 powder prepared according to Reference Example 8 by spray impregnation was further processed with boehmite (Pural SB; Sasol) as a binder to give extrudates. The amounts of starting materials were chosen such that the extrudates contain 10 weight-% Al₂O₃ as binder.

120 g of zeolite powder were weighed in a kneader and kneaded for 5 minutes. Separately, a suspension was prepared of 17.78 g of boehmite (Pural SB; Sasol) in 80 g of deionized water. To that suspension, 3.18 g aqueous nitric acid (65 weight-% of nitric acid in deionized water) were admixed and the resulting suspension was stirred for 1 minute such that a gel was formed. Then, the formed gel was added to the zeolitic material in the kneader and the resulting mixture was kneaded for 30 minutes. 2.76 g of a polysaccharide (Zusoplast PS1) and 0.69 g of polyethylene oxide (PEO E160) were added and the resulting mixture kneaded for 5 minutes. A portion of 10 g of water was subsequently admixed and the resulting mixture was kneaded for 2 minutes. Then, a portion of 1 g of water was admixed and the resulting mixture was kneaded for 2 minutes. The kneaded material was pressed with the aid of an extrudate press through a 2.5 mm die at a pressure of 93 to 148 bar. Subsequently, the resulting extrudates were dried in a circulating oven at a temperature of 120° C. for 4 h (whereby the heating ramp was set to 2° C./min) and then calcined in a static oven at 550° C. for 5 h (whereby the heating ramp was set to 2° C./min). The extrudates may be broken manually to a desired length.

The resulting extrudates had a crush strength of 17.1 N. The Mg content of the resulting extrudates was 1.9 g Mg/100 g, the Al content was 5.9 g/100 g, the Si content was 38 g/100 g, the C content was less than 0.1 g/100 g, and the BET specific surface area was 354 m²/g. Further, the resulting extrudates displayed a total pore volume of 0.49 g/ml. In addition, the acid site density was determined according to NH₃-TPD as disclosed herein as being 0.286 mmol/g at a temperature of below 250° C. and 0.261 mmol/g at a temperature in the range of from greater than 250° C. to 650° C.

It can be gathered from the results for the determination of the mechanical strength that the novel molding prepared according to the present invention shows a comparatively higher crush strength than a molding prepared according to the prior art while having a similar composition. In particular, it has been shown that a molding prepared according to Example 12 of the present invention shows a crush strength of 17.1 N whereas a molding in accordance with the prior art shows a crush strength of 5.5 N.

### Example 13: Catalytic Testing—Methanol-to-Olefin Reaction

A methanol-to-olefin (MTO) reaction was carried out at a temperature of 490° C. and at a pressure of 4 bar(gauge) in a fixed-bed reactor. A sample (2.7 g, 1.6-2.0 mm sieve fraction) was heated in flowing nitrogen (10 NI/h) at 490° C. in 3 h. A feed stream containing 20 volume-% methanol, 70 volume-% water, and 10 volume-% nitrogen was continuously fed into the catalyst bed with a weight hourly space velocity (WHSV) of 10 h⁻¹ and a gas hourly space velocity (GHSV) of 20224 h⁻¹. The time on stream was about 70 h. The products were analyzed by online gas chromatography (Agilent 7890A) with a TCD detector, 2 FID detectors, and using Select Permanent CO₂ HR, Restek Stabilwax and Al₂O₃ MAPD columns.

The methanol conversion X was calculated according to formula II:

X=1−(MeOHₒᵤₜ/MeOHᵢₙ) (II)

In formula II MeOHₒᵤₜ is the methanol at the outlet of reactor and MeOHᵢₙ is the methanol at the inlet of reactor.

The selectivity of the different products is given according to formula III as:

$${S}_{i} = \frac{( {NC}_{i ( out )} \cdot {n}_{i ( out )} ) - ( {NC}_{i ( i n )} \cdot {n}_{i ( i n )} )}{{\sum }_{i} [ ( {NC}_{i ( out )} \cdot {n}_{i ( out )} ) - ( {NC}_{i ( i n )} \cdot {n}_{i ( i n )} ) ]} \qquad ( III )$$

In formula III, NCᵢ is number of carbon atoms in the component i, nᵢ is the number of moles of the component i, (out) refers to the stream outlet of the reactor, and (in) refers to the inlet stream of the reactor.

A molding prepared according to Comparative Example 9 and a molding prepared according to

Example 10 were tested in the conversion of methanol to olefins.

The results of the catalytic testing are shown in FIGS. 1 and 3 below. As can be gathered from FIG. 2, a molding according to Comparative Example 9 reflecting the prior art displays a methanol conversion in the first 20 h in the range of from 90 to 100%, which drops during the following 40 h to about 80%, and continued decreasing to about 70%. The methanol conversion is strongly fluctuating after 40 h on stream. The selectivity towards olefins was slightly above 70% for the first 40 h, then decreased to a value in the range of from 50 to 68%. The selectivity towards butylenes was about 20% for the whole testing time, the selectivity towards propylene was about 39 to 42% for the first 60 h, then decreased to value in the range of from 30 to 40%, and the selectivity towards ethylene was about 6 to 10% for the whole testing time.

In contrast thereto, a molding prepared according to Example 10 of the present invention displays a methanol conversion in the range of from 90 to 100% for the first 40 h, and remained at about 90% for the following 20 h, before slightly decreasing to a value in the range of from 80 to 90%. The selectivity towards olefins was slightly above 70% for the first 40 h, then decreased slightly to a value in the range of from 60 to 70%. The selectivity towards butylenes was about 20% for the whole testing time, the selectivity towards propylene was about 39 to 45% for the whole testing time, and the selectivity towards ethylene was about 6 to 10% for the whole testing time.

Further, the moldings prepared according to Examples 11 and 12, respectively, of the present invention display a methanol conversion in the range of from 90 to 100% for the first 50 h, and remained at about 90% for the following 30 h. The selectivity towards olefins was approximately 70% over the whole testing period. The selectivity towards butylenes was about 20% for the whole testing time, the selectivity towards propylene was about 39 to 45% for the whole testing time, and the selectivity towards ethylene was about 7 to 10% for the whole testing time.

Therefore, it can be gathered from the results for catalytic testing that a molding according to the present invention achieves an overall superior performance not only with respect to the level of conversion of methanol and the particular selectivity towards the desired olefins, but also with respect to the long-term performance. Thus, as can be gathered from the results a molding of the present invention shows longer catalytic activity on a comparatively higher level.

### DESCRIPTION OF FIGURES

FIG. 1: shows a double logarithmic plot of data from a catalyst support at the various diffusion times used. On the ordinate, the signal is given in arbitrary units and on the abscissa, the value b is given. The slope of each line corresponds to a diffusion coeffi-cient.

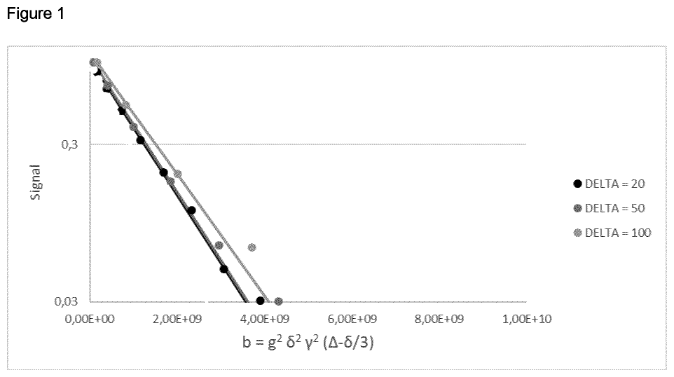

FIG. 2: shows the catalytic performance of a molding prepared in accordance with Comparative Example 9. On the abscissa, the time on stream (TOS) is given in hours and on the ordinate, the conversion relative to methanol as well as the selectivity towards olefins, butylenes, propylene, ethylene, alkanes, and carbon oxides (CO and CO₂) is given in %, determined according to Example 12.

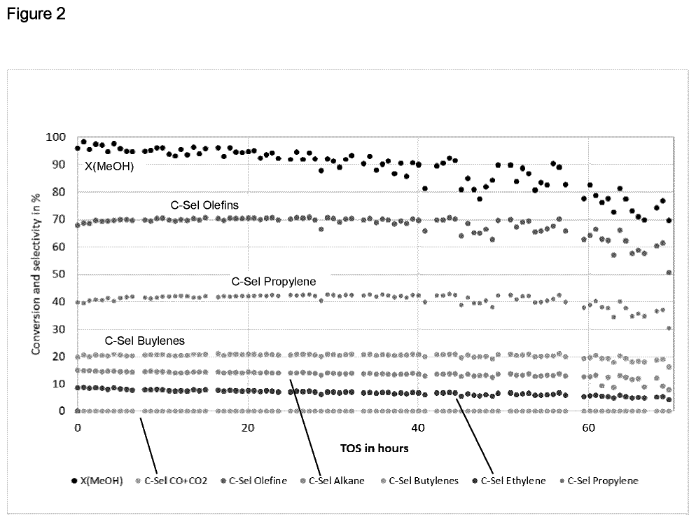

FIG. 3: shows the catalytic performance of a molding prepared in accordance with Example 10. On the abscissa, the time on stream (TOS) is given in hours and on the ordinate, the conversion relative to methanol as well as the selectivity towards olefins, butylenes, propylene, ethylene, alkanes, and carbon oxides (CO and CO₂) is given in %, determined according to Example 12.

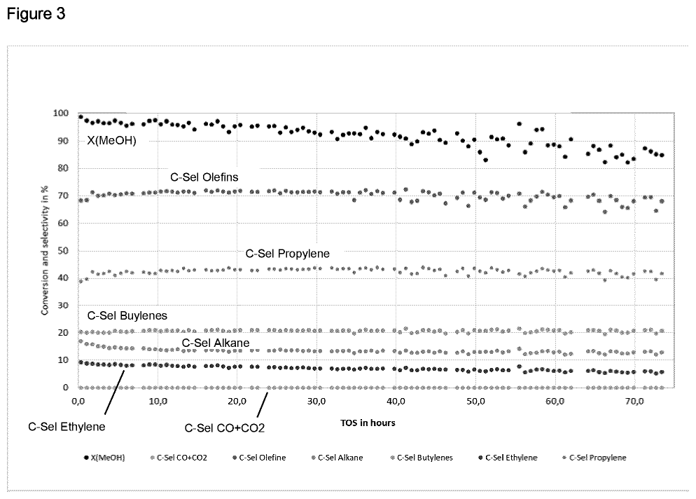

FIG. 4: shows the catalytic performance of a molding prepared in accordance with Example 11. On the abscissa, the time on stream (TOS) is given in hours and on the ordinate, the conversion relative to methanol as well as the selectivity towards olefins, butylenes, propylene, ethylene, alkanes, and carbon oxides (CO and CO₂) is given in %, determined according to Example 12.

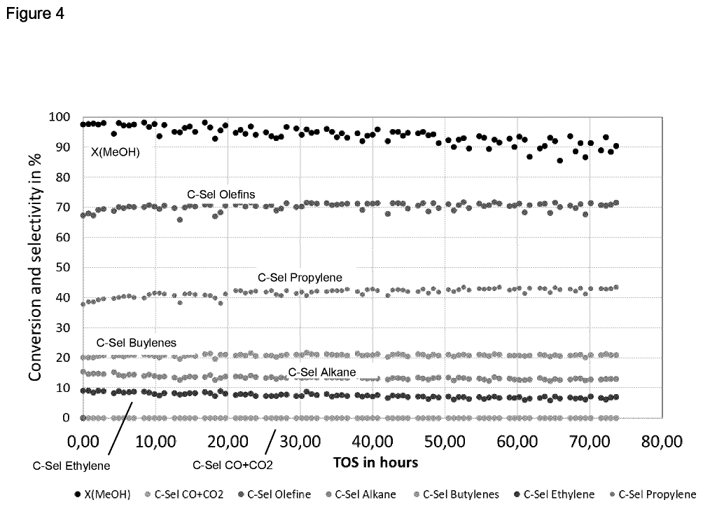

FIG. 5: shows the catalytic performance of a molding prepared in accordance with Example 12. On the abscissa, the time on stream (TOS) is given in hours and on the ordinate, the conversion relative to methanol as well as the selectivity towards olefins, butylenes, propylene, ethylene, alkanes, and carbon oxides (CO and CO₂) is given in %, determined according to Example 13.

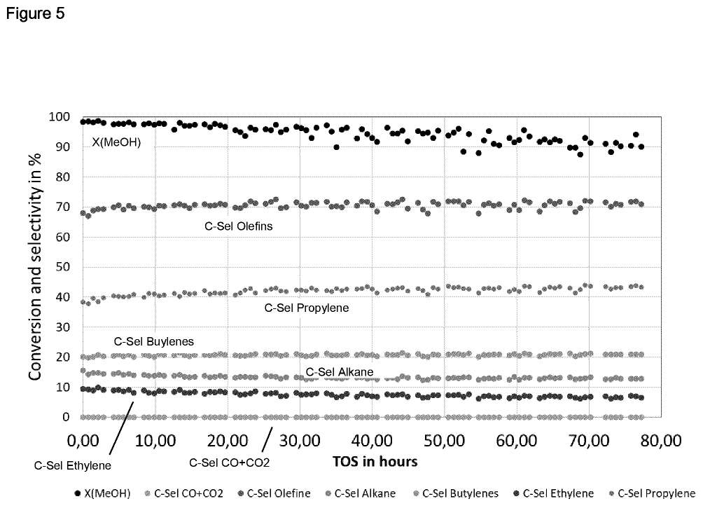

### CITED LITERATURE

WO 2012/085154 A1

US 2014/0058180 A1

U.S. Pat. No. 10,112,188 B2

US 2014/0058181 A1

U.S. Pat. No. 10,005,073 B2

U.S. Pat. No. 9,511,361 B2

US 2017/0121259 A1

WO 2018/109083 A1

CN 100503041 C

CN 104511298 B

1-15. (canceled)

16. A process for preparing a molding comprising a zeolitic material and one or more oxidic binders, wherein the zeolitic material comprises YO₂ and X₂O₃ in its framework structure, wherein Y is Si and X is a trivalent element, the process comprising
(i) preparing a mixture comprising a zeolitic material, a source of an oxidic binder, a first plasticizer, and an acid;
(v) shaping of the mixture obtained in (i), to obtain a precursor of a molding;
wherein in the mixture prepared in (i) the weight ratio of the source of the oxidic binder, calculated as the oxide, to the sum of the zeolitic material and the source of the oxidic binder, calculated as the oxide, is in the range of from 0.05:1 to 0.15:1;
wherein the source of the oxidic binder comprises one or more of AlOOH (boehmite) and Al₂O₃; wherein X is selected from the group consisting of B, Al, Ga, In, and a mixture of two or more thereof.

17. The process of claim 16, wherein (i) comprises
(i.1.a) preparing a mixture comprising a zeolitic material, a source of an oxidic binder, and a first plasticizer;
(i.1.b) admixing an acid to the mixture obtained in (i.1.a);
wherein the acid is admixed in (i.1.b), comprising an amount of the acid in the range of from 5 to 50 weight-%; and wherein the weight ratio of the acid admixed in (i.1.b) to the sum of the zeolitic material and the source of the oxidic binder of the mixture prepared in (i) is in the range of from 0.05:1 to 0.15:1.

18. The process of claim 16, wherein (i) comprises
(i.2.a) providing a zeolitic material;
(i.2.b) providing a mixture comprising a source of an oxidic binder, optionally water, and an acid;
(i.2.c) mixing the mixture obtained in (i.2.b) with the zeolitic material provided in (i.2.a);
(i.2.d) admixing a first plasticizer to the mixture obtained in (i.2.c);
wherein the acid is provided for the mixture of (i.2.b) as an aqueous solution comprising an amount of the acid in the range of from 50 to 80 weight-%,
wherein the weight ratio of the acid provided for the mixture of (i.2.b) to the sum of the zeolitic material and the source of the oxidic binder of the mixture prepared in (i) is in the range of from 0.005:1 to 0.05:1.

19. The process of claim 16, wherein the first plasticizer is selected from the group consisting of organic polymers, carbohydrates, graphite, plant additives, and mixtures of two or more thereof.

20. The process of claim 16, wherein the source of the oxidic binder comprises AlOOH (boehmite).

21. The process of claim 16, wherein the zeoltic material has a framework structure type selected from the group consisting of ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE, BSV, CAN, CAS, CDO, CFI, CGF, CGS, CHA, -CHI, -CLO, CON, CSV, CZP, DAC, DDR, DFO, DFT, DOH, DON, EAB, EDI, EEI, EMT, EON, EPI, EM, ESV, ETR, EUO, *-EWT, EZT, FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH, *-ITN, ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MEI, MFS, MON, MOR, MOZ, *MRE, MSE, MSO, MTF, MTN, MTT, MTW, MVY, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF, *-SSO, SSY, STF, STI, *STO, STT, STW, -SVR, SVV, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, and mixed types of two or more thereof.

22. The process of claim 16, wherein the zeolitic material comprises one or more alkaline earth metals M.

23. The process of claim 16, wherein the acid is one or more of an inorganic acid and an organic acid.

24. The process of claim 16, wherein in (v), the mixture is shaped to a strand.

25. A molding obtainable or obtained by the process of claim 16.

26. A molding comprising one or more oxidic binders and a zeolitic material, wherein the zeolitic material comprises YO₂ and X₂O₃ in its framework structure,
wherein Y is Si and X is a trivalent element,
wherein the molding comprises the one or more oxidic binders, calculated as the oxide, in an amount in the range of from 5 to 15 weight-%, and wherein the molding exhibits a crush strength of equal or greater than 9 N, determined according to Reference Example 5, wherein the one or more oxidic binders are alumina, wherein X is selected from the group consisting of B, Al, Ga, In, and a mixture of two or more thereof.

27. The molding of claim 26, exhibiting a diffusion coefficient in the range of from 0.40 to 1.30×10⁻⁹ m²/s, determined according to Reference Example 4.

28. The molding of claim 26, exhibiting a tortuosity parameter relative to water in the range of from 1.00 to 3.75, determined as described in Reference Example 2.

29. A method for the conversion of oxygenates to olefins comprising
(a) providing a molding according to claim 25;
(b) providing a gas stream comprising one or more oxygenates and optionally one or more olefins and/or optionally one or more hydrocarbons
(c) contacting the molding provided in (a) with the gas stream provided in (b) and converting one or more oxygenates to one or more olefins and optionally to one or more hydrocarbons;
(d) optionally recycling one or more of the one or more olefins and/or of the one or more hydrocarbons contained in the gas stream obtained in (c) to (b).

30. Use of a molding according to claim 25 as a molecular sieve, as an adsorbent, for ion-exchange, or as a catalyst and/or as a catalyst support.
